(19) United States
(12) Patent Application Publication
(10) Pub. No.: US 2022/0154192 A1
(43) Pub. Date: May 19, 2022

(54) SYSTEM AND METHOD FOR LIGHT-REGULATED OLIGOMERIZATION AND PHASE SEPARATION OF FOLDED DOMAINS AND RNA GRANULE-ASSOCIATED PROTEIN DOMAINS FOR DRUG- BASED SCREENING APPLICATIONS

(71) Applicant: The Trustees of Princeton University, Princeton, NJ (US)

(72) Inventors: Clifford P. Brangwynne, Hopewell, NJ (US); Dan Bracha, Princeton, NJ (US); Victoria Drake, Quacker Hill, CT (US); David W. Sanders, Princeton, NJ (US)

(73) Assignee: The Trustees of Princeton University, Princeton, NJ (US)

(21) Appl. No.: 17/432,145

(22) PCT Filed: Feb. 19, 2020
(86) PCT No.: PCT/US2020/018758

§ 371 Date: Aug. 19, 2021

Related U.S. Application Data
(60) Provisional application No. 62/807,459, filed on Feb. 19, 2019.

(51) Int. Cl. C12N 15/62; C12N 15/113; C12N 9/22; C12N 15/63

(52) U.S. Cl. CPC C12N 15/62; C12N 15/635; C12N 9/22; C12N 15/113

(57) ABSTRACT

Disclosed is a method and system for the phase separation of folded domains, and more particularly, to inducing clusters of folded domains as part of a drug-based screening application. The system and method utilize one or more first fusion proteins (100, 101), each first fusion protein comprising a first region (110) fused to a second region (120), the first region (110) comprising at least one light sensitive protein (115) or cognate partner of a light sensitive protein (116), and the second region (120) comprising one or more folded RNA binding domains (RBDs), disordered RBDs, folded non-RBD domains, or combination thereof (125).

### CROSS REFERENCE TO RELATED APPLICATIONS

This application claims priority to U.S. Provisional Application No. 62/807,459, filed Feb. 19, 2019, which is herein incorporated by reference in its entirety.

### TECHNICAL FIELD

The present disclosure relates generally to the phase separation of folded domains, and more particularly, to inducing clusters of folded domains as part of a drug-based screening application.

### BACKGROUND

Cells compartmentalize the diverse biochemical processes necessary for life in reaction centers called organelles. Organelles are classically depicted as membrane-enclosed compartments sequestered from a homogenous cytosolic solution. However, cells also organize their contents with organelles lacking membranes. Such compartments are particularly abundant in the nuclei of eukaryotic cells and include ribosome-producing nucleoli as well as RNA-protein bodies of poorly understood function (e.g. Cajal bodies, speckles) (Zhu and Brangwynne, 2015). In the cytoplasm, the presence of membraneless compartments is usually context-specific, appearing as a consequence of polysome disassembly (i.e. stress granules) (Ivanov et al., 2018; Protter and Parker, 2016) or the detection of specific extracellular signals (e.g. signalosomes, inflammasomes) (Gammons and Bienz, 2018; Wu and Fuxreiter, 2016). In many cases, it is unclear whether such macroscopic assemblies augment specific biochemical reactions or exist passively as inert sequestration centers (Shin and Brangwynne, 2017).

Recent studies suggest that the physics of liquid-liquid phase separation (LLPS) accounts for the assembly of these structures, which are increasingly referred to as condensates (Banani et al., 2017; Brangwynne et al., 2009; Shin and Brangwynne, 2017). Intracellular LLPS occurs at saturating protein/nucleic acid concentrations as a consequence of free energy minimization through preferential self-associations (Brangwynne et al., 2015). Although weak interactions between proteins containing low-complexity/intrinsically disordered regions (IDRs) or short “sticky” motifs can mediate intracellular LLPS in certain systems (Elbaum-Garfinkle et al., 2015; Frey et al., 2006; Kato et al., 2012; Molliex et al., 2015; Murakami et al., 2015; Nott et al., 2015; Patel et al., 2015), it is unclear whether these additive “multivalent” motif repeats are essential for the formation of common, macroscopic biological condensates like nucleoli and stress granules. In such RNP bodies, proteins containing low-specificity RNA-binding domains (RBDs) may be critical for LLPS, due to their weak interactions with RNA-based cross-links (Chong et al., 2018; Feric et al., 2016; Lee et al., 2016; Mitrea et al., 2018; Nott et al., 2015; Vernon et al., 2018).

Despite the large number of studies on the contributions of multivalent weak interactions, less attention has been paid to specific interactions that allow multi-component cellular condensates to selectively phase separate and recruit certain substrates while excluding others. Essential condensate-nucleating proteins often exhibit a shared modular structure that includes oligomerization domains, IDRs, and substrate-binding moieties, the most common category being RBDs (Aoki et al., 2018; Hebert and Matera, 2000; Kedersha et al., 2016; Matsuki et al., 2013; Mitrea et al., 2018; 2016; 2014; Tourriere et al., 2003). Many of these RBDs feature both a well-folded RNA recognition motif (RRM) that binds with high-affinity to specific RNA motifs and a terminal RGG region, which binds with low-affinity to bulk RNA and dissociated ribosomes (Chong et al., 2018; Mitrea et al., 2016; Thandapani et al., 2013). For example, G3BP (stress granules), PGL (P granules), and NPM1 (nucleoli), each have an oligomerization domain on the N-terminus and a bi-partite RBD (folded RRM, disordered RGG) on the C-terminus (Aoki et al., 2018; Kedersha et al., 2016; Matsuki et al., 2013; Mitrea et al., 2014; Tourriere et al., 2003). While such oligomerization domains and RBDs are thought to be important, we lack a quantitative understanding of their relative contributions to phase separation of condensates with defined material properties.

Stress granules (SGs) represent a particularly interesting cytoplasmic condensate, which has emerged as an important model for elucidating general principles of intracellular phase separation (Ivanov et al., 2018; Kedersha et al., 1999; Protter and Parker, 2016). SGs are micron-sized, liquid-like RNA-protein assemblies that form in mammalian cells in response to translational arrest and subsequent polysome disassembly (Kedersha et al., 1999; 2016; 2002; Kroschwald et al., 2015; Molliex et al., 2015; Wheeler et al., 2016; Wippich et al., 2013). SG assembly involves a network of interacting RBPs, ribosomal subunits, and RNAs (Bounedjah et al., 2014; Kedersha et al., 2016; Markmiller et al., 2018; Youn et al., 2018). Despite this rich network of interactions, previous work has underscored the importance of a single protein, G3BP, which appears to be essential for SG condensation (Kedersha et al., 2016; Matsuki et al., 2013), and features the canonical modular architecture described above (Tourriere et al., 2003). However, the mechanism by which G3BP regulates SG biogenesis, and the biophysical role of its modular architecture, remain poorly understood (Kedersha et al., 2016; Matsuki et al., 2013; Panas et al., 2015; Schulte et al., 2016; Solomon et al., 2007; Wu et al., 2016).

A key obstacle hindering previous work on SGs and other condensates has been a lack of tools to quantitatively probe the relative roles of unique interaction modules for specific condensate nucleating proteins in vivo.

### BRIEF SUMMARY

Phase separation/condensation generally requires the formation of connected network of interacting biomolecules. The disclosed system and method allow one of skill in the art to engineer constructs that activate phase separation upon light activation, but only if potential protein-protein or protein-RNA interactions are occurring. This in turn allows one to screen for conditions which disrupt said interactions, by finding conditions under phase separation/condensation does not occur, due to the loss of a connected network of interactions.

Disclosed is a system and method that provide an optogenetic tool to quantitatively examine biomolecular interactions, for example oligomerization, protein-protein interactions, and RNA-binding, utilizing intracellular condensation as a read-out. A weakly cross-linked complex or hub of folded domains can be used to surpass the phase boundary for liquid-liquid phase separation. These hubs may be disrupted by, e.g., a molecule from a small molecule library, or a physiological protein/substrate such as USP10, which decrease the complex's valence and thereby abrogates its ability to mediate phase separation of the associated protein-RNA network.

A first aspect of the present disclosure is drawn to a protein system, which can be used as part of a drug-based screening application. The protein system requires one or more first fusion proteins, where each first fusion protein includes a first region fused to a second region. The first region comprises at least one light sensitive protein or cognate partner of a light sensitive protein, while the second region comprises one or more folded RNA binding domains (RBDs), disordered RBDs, folded non-RBD domains, or combination thereof.

Optionally, when the first region of the first fusion protein includes a first cognate partner of a first light sensitive protein, the system may include a second fusion protein, which also includes a first region fused to a second region. The first region of the second fusion protein includes the first light sensitive protein (allowing the first fusion protein to connect to the second fusion protein under appropriate light conditions). The second region of the second fusion protein includes one or more folded RNA binding domains (RBDs), disordered RBDs, folded non-RBD domains, or combination thereof, where the second region of the second fusion protein is capable of self-assembly (e.g., via dimer, trimer, pentamer, n-mer interactions, including homotypic and heterotypic interactions) when near other second fusion proteins.

Further, such a system may also include a third and a fourth fusion protein, where the second and fourth fusion proteins self-assemble into a core structure, and the first and third fusion proteins are configured to interact with each other and to be optogenetically attachable to the second and fourth fusion proteins, respectively. The third fusion protein includes a first region fused to a second region. The first region of the third fusion protein comprises a cognate partner of a second light sensitive protein, and the second region of the third fusion protein comprising one or more folded RNA binding domains (RBDs), disordered RBDs, folded non-RBD domains, or combination thereof, where the second region of the third fusion protein is adapted to interact with the second region of the first fusion protein. The fourth fusion protein includes a first region fused to a second region, the first region of the fourth fusion protein comprising the second light sensitive protein, and the second region of the fourth fusion protein comprising one or more folded RNA binding domains (RBDs), disordered RBDs, folded non-RBD domains, or combination thereof, where the second region of the fourth fusion protein is capable of self-assembly, either with other fourth fusion proteins or with other second fusion proteins.

Alternatively, the system could include a third fusion protein having two regions fused together, each region comprising one or more folded RNA binding domains (RBDs), disordered RBDs, folded non-RBD domains, or combination thereof, and each of the two regions of the third fusion protein being adapted to interact with the second region of the first fusion protein.

Optionally, when the first region of the first fusion protein comprises a first light sensitive protein, the system may also include a second fusion protein and two or more third fusion proteins. The second fusion protein includes a first region fused to a second region, where the first region of the second fusion protein utilizes a cognate partner of the first light sensitive protein, and the second region of the second fusion protein is identical to the second region of the first fusion protein. That is, the first and second fusion proteins are near-identical, saving that one has a light sensitive protein, and one has a cognate partner of the light sensitive protein. The two or more third fusion proteins each include a first region fused to a second region, each of the two regions of the third fusion protein include one or more folded RNA binding domains (RBDs), disordered RBDs, folded non-RBD domains, or combination thereof, the second region of the third fusion protein comprising one or more folded RNA binding domains (RBDs), disordered RBDs, folded non-RBD domains, or combination thereof, but the first region is adapted to interact with the second region of the first fusion protein or the second region of second fusion protein, and the second region of each third fusion protein is adapted to self-assemble.

Optionally, when the first region of the first fusion protein/optoprotein comprises a first light sensitive protein, the system may utilize a second fusion protein including a first region fused to a second region. The first region of the second fusion protein includes the first light sensitive protein, and the second region of the second fusion protein comprising one or more folded RNA binding domains (RBDs), disordered RBDs, folded non-RBD domains, or combination thereof, where the second region of each second fusion protein is adapted to interact with the second region of the first fusion protein, and where the first region of the first fusion protein and the first region of the second fusion protein are adapted to self-assemble in response to light into an oligomer of at least 2.

In some of the disclosed systems, the light-sensitive protein is fused to a folded RBD, and the folded RBD is an RNA recognition motif (RRM), a K homology (KH) domain, a Pumilio (PUM) domain, a zinc-finger domain, a DEAD box helicase domain, a double-stranded RNA-binding domain (dsRBD), an m6A RNA-binding domain (YTH domain), or a Cold shock domain (CSD). In some of the disclosed systems, the light-sensitive protein is fused to a disordered RBD, and the disordered RBD is an arginine-glycine (RG) domain, an arginine-glycine-glycine (RGG) domain, a serine-arginine (SR) domain, or a basic-acidic dipeptide (BAD) domain (e.g. RD, RE). In some of the disclosed systems, the light-sensitive protein is fused to one or more folded non-RBDs.

In some of the disclosed systems, the first region comprises ferritin.

Optionally, the at least one light-sensitive protein is an engineered protein, such as LOV2-ssrA. Optionally, the at least one light-sensitive protein comprises a first LOV2-ssrA fused to a second LOV2-ssrA.

Optionally, one of the fusion proteins in the system, such as the first fusion protein, comprises a fluorescent tag.

A second aspect of the present disclosure is drawn to a cell line or stem cell-derived cell that expresses the protein system described above. Optionally, one or more genes configured to express the protein system were delivered to the cells utilizing a lentivirus, an adeno-associated virus (AAV), bacterial artificial chromosomes (BAC), transient transfection (e.g. liposomes or proprietary formulations for DNA plasmid introduction), micro-injection, electroporation, or a CRISPR/Cas9-based approach. Optionally, the cells are human cells, yeast cells, cultured neurons, or worm, fly, rodent, or primate models.

A third aspect of the present disclosure is drawn to an expression vector system comprising at least one expression vector configured to transfect a cell with one or more genes configured to express the protein system according to claim 1. Optionally, the expression vector system includes a first plasmid comprising a gene capable of expressing the first fusion protein.

A fourth aspect of the present disclosure is drawn to a method for measuring phase behavior (i.e., a concentration-dependent phase diagram, including saturation concentration, full binodal phase boundary, etc.) of natural or engineered multi-component membraneless organelles/condensates. The method includes providing a protein system described above, oligomerizing the folded RNA binding domain (RBD), disordered RBD, or folded non-RBD domains by exposing the light-sensitive protein to at least one wavelength of light, and measuring phase behavior by mapping a phase diagram, determining if phase separation, condensation, or aggregation occurs, measuring a condensate material property, a protein concentration, a valence, or a combination thereof.

Optionally, the method can be when the protein system is located within a living cell, or outside a living (or dead) cell. Optionally, the protein system is in a well in a multi-well array (or plate). Optionally, oligomerization drives gelation of a cytoplasmic ribonucleoprotein (RNP) granule.

Optionally, the method also includes providing one or more chemical agents to the well.

Optionally, the method also includes utilizing a genetic screen based on gene knockdown (e.g., CRISPR KO, CRISPRi, siRNA, shRNA, or antisense oligonucleotides) or gene upregulation (e.g., CRISPRa or DNA plasmid-based overexpression).

Optionally, the method also includes determining the impact a genetic screen based on gene knockdown, a genetic screen based on upregulation, the addition of one or more chemical agents to a well, or a combination thereof has, based on the measured phase behavior.

### BRIEF DESCRIPTION OF THE DRAWINGS

FIG. 1A is a simplified embodiment of a first fusion protein according to the present disclosure, highlighting the first and second regions of the fusion protein.

FIG. 1B is a simplified alternate embodiment of a first fusion protein according to the present disclosure, highlighting the first and second regions of the fusion protein.

FIG. 1C is a simplified diagram illustrating an embodiment where a single type of first fusion protein can self-assemble.

FIG. 2 is a simplified embodiment of a second fusion protein according to the present disclosure.

FIG. 3 is a simplified diagram illustrating an embodiment where the second fusion proteins self-assemble, and the first fusion protein can attach to the self-assembled core under certain wavelengths of light.

FIG. 4 is a simplified embodiment of a fifth fusion protein according to the present disclosure.

FIG. 5 is a simplified diagram illustrating an embodiment where the second fusion proteins self-assemble, the first fusion protein can attach to the second fusion proteins under certain wavelengths of light, and the third fusion protein interacts with the second fusion protein.

FIG. 6 is a simplified embodiment of a third fusion protein according to the present disclosure.

FIG. 7 is a simplified embodiment of a fourth fusion protein according to the present disclosure.

FIG. 8 is a simplified diagram illustrating an embodiment where the second and fourth fusion proteins self-assemble, the first fusion protein can attach to the second fusion proteins under certain wavelengths of light, the third fusion protein interacts with the second fusion protein, and the third fusion protein can attach to the fourth fusion proteins under certain wavelengths of light.

FIG. 9 is a simplified embodiment of a sixth fusion protein according to the present disclosure.

FIG. 10 is a simplified diagram illustrating an embodiment where the sixth fusion proteins self-assemble, one type of first fusion protein can interact with the sixth fusion protein, a second type of first fusion protein can attach to the first type of first fusion protein under certain wavelengths of light, and the second type of first fusion protein is available to interact with a sixth fusion protein.

FIG. 11 is a simplified diagram illustrating an embodiment where different types of first fusion proteins can self-assemble.

FIG. 12 is an illustration showing how concepts from graph theory inform a mechanistic framework for network-based cellular condensation, concepts which underlie the present application; “Valence” (v) describes the number of interaction sites associated with a “particle” (shown: 1 to 6), which in the cell is an individual protein or protein complex (a “cap” refers to a particle with ν=1; “bridge”, ν=2; “node”, ν>2), and an assembled complex results from connections between individual particles, each featuring their own valence (indicated). A particle that lacks interactions with the larger complex is a “bystander” (ν=0). If viewed in isolation (e.g. RBP complex, no RNA), the complex features an overall valence (here, ν=6) reflecting the number of available sites for additional connectivity (e.g. RNA-binding domains). In the context of G3BP-mediated SGs (bottom), in non-stressed cells the amount of exposed mRNA for G3BP-binding is typically low (high occupancy of ribosomes); following arsenite stress-induced polysome disassembly, mRNA is exposed and network condensation is mediated by high RBD valence of the G3BP node.

FIG. 13 are Western blots from GFP-tagged G3BP domain deletion co-immunoprecipitation studies that validate endogenous protein interaction partners predicted by the described technology using a folded domain of G3BP (NTF2), where the legend shows the various domains for G3BP (1300), as similarly seen in G3BP1, G3BP2A and G3BP2B (1301, 1302, 1303): an oligomerization domain (NTF2 (dimerization): 1-141 (1310); two IDR domains (IDR1 (acidic) (142-224) (1320) and IDR2 (P-rich) (225-334) (1330)) and two RBD domains (RRM domain (334-409) (1340) and RGG domain (410-466) (1350) (recognizing that different isoforms have the same domain organization, but different amino acid designations)). Deletion of NTF2 domain (1305) abolishes stress-independent, high affinity binding of GFP-G3BP to USP10, CAPRIN1, and UBAP2L in G3BP KO (RNAse, RIPA wash of beads). Representative Western blot from three independent experiments.

FIG. 14A is a simplified depiction of five domains of interest in G3BP (1400): an oligomerization domain (NTF2 (dimerization): 1-141 (1401); two IDR domains (IDR1 (acidic) (142-224) (1402) and IDR2 (P-rich) (225-334) (1403)) and two RBD domains (RRM domain (334-409) (1404) and RGG domain (410-466) (1405)).

FIG. 14B is a simplified depiction of a sspB-ΔNTF2 (1450), where the NTF2 domain (1401) of G3BP (1400) has been replaced with sspB (1451) but otherwise remains unchanged.

FIG. 14C is a simplified depiction of protein (1460) for screening for dimerization domain (NTF2)-interacting proteins and those that modulate its condensation, containing four domains: an oligomerization domain (NTF2 (dimerization): 1-141 (1401); two IDR domains (IDR1 (acidic) (142-224) (1402) and IDR2 (P-rich) (225-334) (1403)) and sspB (1451).

FIG. 15A is an intracellular phase diagram revealing interplay between core valence, core concentration, and substrate (RNA) availability, where calculated best-fit phase threshold displayed, for an untreated system, where the system uses the same sspB construct as FIG. 14B, and where the experiments are performed in human U2OS cells.

FIG. 15B is an intracellular phase diagram revealing interplay between core valence, core concentration, and substrate (RNA) availability, where calculated best-fit phase threshold displayed, for a system treated with arsenite (available RNA increases), where the system uses the same sspB construct as FIG. 14B, and where the experiments are performed in human U2OS cells.

FIG. 15C is an intracellular phase diagram revealing interplay between core valence, core concentration, and substrate availability, where calculated best-fit phase threshold displayed, for a system treated with arsenite and cycloheximide (Mocks arsenite-induced RNA increase), where the system uses the same sspB construct as FIG. 14B, and where the experiments are performed in human U2OS cells. The calculated best-fit phase threshold is almost identical to that of nontreated cells (FIG. 15A).

FIG. 15D is an intracellular phase diagram for a system using the same sspB construct as FIG. 14B, that is treated with Actinomycin D (decreases available RNA by blocking RNA transcription), revealing the addition of Actinomycin D disrupts the formation of SGs in experiments performed in human U2OS cells.

FIG. 16A is an intracellular phase diagram revealing interplay between core valence and core concentration, where calculated best-fit phase threshold is displayed, where the system uses the same sspB construct as FIG. 14C (i.e. features NTF2 protein-protein interaction domain but lacks RBD), and where the experiments are performed in human U2OS cells.

FIG. 16B is an intracellular phase diagram revealing interplay between core valence, core concentration, and overexpression of a control NTF2-interacting, RNA-binding protein (CAPRIN1-miRFP670), which preserves its network of RNA-binding interactions, where calculated best-fit phase threshold is displayed, where the system uses the same sspB construct as FIG. 14C, and where the experiments are performed in human U2OS cells. The calculated best-fit phase threshold is similar to that of cells expressing no fluorescent protein (FIG. 16A).

FIG. 16C is an intracellular phase diagram revealing interplay between core valence, core concentration, and overexpression of a NTF2-interacting protein (USP10-miRFP670), which disengages its network of RNA-binding protein interactions and inhibits phase separation, where calculated best-fit phase threshold is displayed, where the system uses the same sspB construct as FIG. 14C, and where the experiments are performed in human U2OS cells.

FIG. 17 is a graphical illustration of some of the compositionally overlapping stress granule and P-body protein components revealed by technologies in this disclosure, including an illustration (bottom) of how the network connectivity would result in protein complexes and RNA forming stress granules attached to P-bodies.

FIG. 18 are fluorescence correlation spectroscopy (FCS) calibration curves used to approximate GFP and mCherry cytoplasmic concentrations in U2OS cells in order to determine fusion protein concentrations, valence, and phase boundaries for the technologies enumerated in this application. iLID-GFP and mCherry-sspB were used for calibrations due to lack of expected endogenous binding partners, predicted monomeric state, and common use as tags (note that as used herein, LOV2-SsrA may sometimes be referred to as iLID).

FIG. 19 is a flowchart depicting an embodiment of a screening method.

### DETAILED DESCRIPTION

The present disclosure is drawn to a system and method for light-regulated oligomerization and phase separation of folded domains and RNA granule-associated protein domains, particularly for drug-based screening applications.

The system may involve multiple types of fusion proteins, any or all of which may contain a fluorescent protein. These fusion proteins are configured to work together, while being illuminated with certain wavelengths of light, to oligomerize and network together. This oligomerization and networking (or lack thereof) results in a certain phase behavior, which can be monitored in various conditions and environments (such as when adding various chemical or biological agents) to determine under what conditions or environments the phase behavior can be modified.

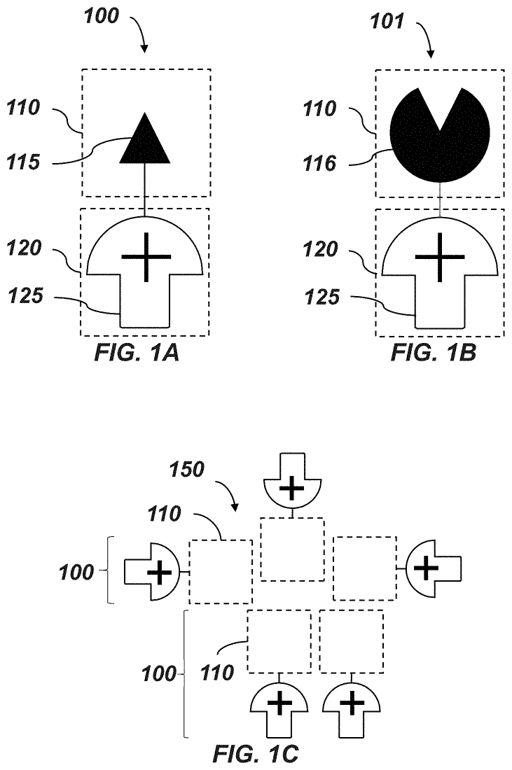

The simplest form of the system can be seen in reference to FIGS. 1A-1C. Referring to FIGS. 1A and 1B, the system requires one or more first fusion proteins (100, 101), sometimes referred to as “optoproteins”, and typically requires a plurality of these first fusion proteins. Each first fusion protein (100, 101) comprises a first region (110) fused to a second region (120).

The first region (110) comprises at least one light sensitive protein (115) or cognate partner of a light sensitive protein (116). These light sensitive proteins (115) or cognate partners (116) can be any light sensitive proteins or cognate partners known to those of skill in the art, including natural or engineered proteins, such as BLUF domains (such as bPAC), Phytochromes (such as Phy-PIF or BphP1-PpsR2), Cryptochromes (such as LARIAT, LITE, OPTOSTIM, Cryptochrome 2 and CIB1), LOV domains (such as BACCS, LAD, LITEZ, iLID [LOV2-SsrA]/SspB, pDawn, and pDusk), Fluorescent protein domains (such as Dronpa based systems and PhoCl), and UVR8 domains (such as UVR8). In one preferred embodiment, the first fusion protein (100) uses a single LOV2-SsrA protein. In another embodiment, the first fusion protein (100) uses two LOV2-SsrA proteins.

As can be seen in the simplified drawings, the cognate partner (116) is adapted to connect with and attach to the light sensitive protein when the light sensitive protein is illuminated with at least one wavelength of light. For example, using the iLID system, when LOV2-SsrA (a light sensitive protein) and SspB (its cognate partner) are mobile within a cell (that is, in a position to be able to interact with each other), the LOV2-SsrA will eventually attach to the SspB only when irradiated with light having approximately a 450 nm wavelength, but will then detach when not irradiated with such light.

The first region (110) may optionally be a region that is configured to self-assemble. For example, in some embodiments, the region comprises one or more proteins that are known to foster self-assembly via dimer, trimer, pentamer, n-mer interactions, including homotypic and heterotypic interactions. In some embodiments, the region comprises a ferritin, which is a family of proteins known to self-assemble into hollow, cage-like structures, each with 24-identical subunits.

The second region (120) comprises one or more folded RNA binding domains (RBDs), disordered RBDs, folded non-RBD domains, or combination thereof (125). Folded RBDs may include, but are not limited to, an RNA recognition motif (RRM), a K homology (KH) domain, a Pumilio (PUM) domain, a zinc-finger domain, a DEAD box helicase domain, a double-stranded RNA-binding domain (dsRBD), an m6A RNA-binding domain (YTH domain), or a Cold shock domain (CSD). Disordered RBDs may include, but are not limited to, an arginine-glycine (RG) domain, an arginine-glycine-glycine (RGG) domain, a serine-arginine (SR) domain, or a basic-acidic dipeptide (BAD) domain (e.g., RD, RE). Folded non-RBDs may be, but are not limited to dimerization or oligomerization domains (e.g., G3BP NTF2, NPM1 oligomerization domain, HSF1 trimerization domain, DCP1A trimerization domain, etc), which are often essential to the formation of physiological biological condensates (e.g., stress granules, nucleoli, nuclear stress bodies, P-bodies, etc). Full length proteins may be used without pre-existing knowledge of oligomerization or substrate-binding (e.g., RNA-binding) domains

If the first fusion protein (100, 101) contains a fluorescent protein, it may be present in either the first region (110) or second region (120), although preferably it is present in the first region.

Referring to FIG. 1C, it can be seen that this basic form of the system (150) can self-assemble upon irradiation with a predetermined wavelength of light (based on the specific light sensitive protein involved) due to the interactions between the first regions (110) of multiple first fusion proteins (100, 101). As can be seen, the system in FIG. 1C has a heterogeneous cluster of first fusion proteins, here shown to include both a first type of first fusion protein (100) as well as an alternative type (101). In other embodiments, the system may form homogeneous clusters.

More complex systems are required when the first fusion proteins (100, 101) do not self-assemble when irradiated with an appropriate wavelength of light.

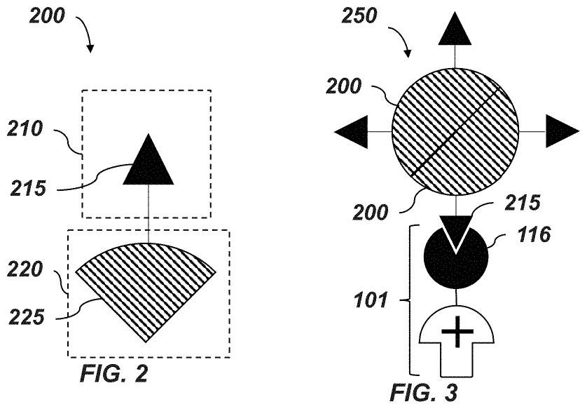

Referring to FIGS. 2 and 3, a first option is to introduce a second fusion protein (200), sometimes referred to as a “core protein”. The second fusion protein (200) also includes a first region (210) fused to a second region (220). The first region (210) includes a light sensitive protein (215). The second region (220) comprises one or more folded RNA binding domains (RBDs), disordered RBDs, folded non-RBD domains, or combination thereof (225), the second region of the second fusion protein being adapted to self-assemble, via dimer, trimer, pentamer, or n-mer interactions, including homotypic and heterotypic interactions. In some embodiments, the region comprises a ferritin, which is a family of proteins known to self-assemble into hollow, cage-like structures, each with 24-identical subunits.

FIG. 3 illustrates a system (250) with first (101) and second (200) fusion proteins. When a plurality of second fusion proteins/core proteins (200) are in a system that allows the fusion proteins to interact, the light sensitive protein (215) of a second fusion protein/core protein (200) can attach to the cognate partner of the light sensitive protein (116) that is present on a first fusion protein/optoprotein (101).

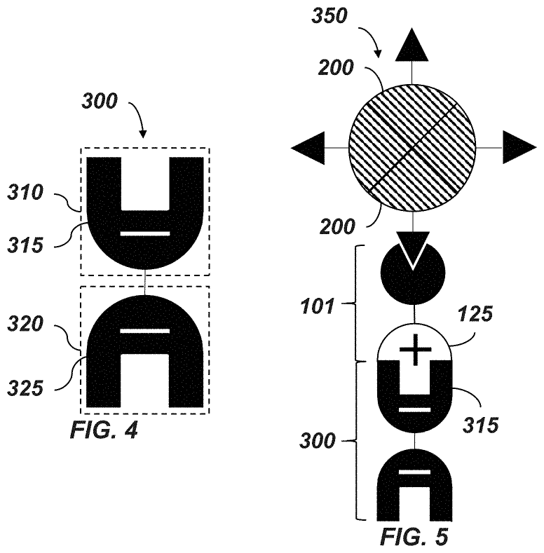

Referring to FIGS. 4-5, a second option is to build on the first option, by introducing a third fusion protein (300). The third fusion protein is sometimes referred to as a “fixed linker”. It can connect versions of the system like those in FIG. 3, allowing for significantly more interactions and larger networks. The third fusion protein (300) comprises at least two regions—a first and second region (310, 320)—fused together. For the third fusion protein, each region comprises one or more folded RNA binding domains (RBDs), disordered RBDs, folded non-RBD domains, or combination thereof (315, 325), and each first and second region (310, 320) of the third fusion protein is adapted to interact with the second region of the first fusion protein.

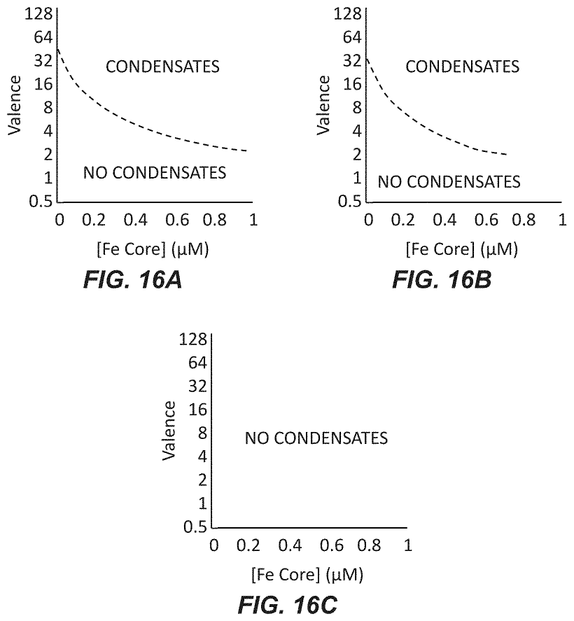

Referring to FIG. 5, one preferred embodiment of the presented system is a dimerization or higher-order oligomerization domain that requires an addition endogenous protein that is adapted to interact for phase separation to occur. For instance, in the G3BP NTF2 (dimerization domain) example, such an endogenous protein is UBAP2L, which allows further networking between G3BP dimers and condensation. Removal of the protein from cells via knockout or over-expression of a protein, USP10, that competes for its interactions at the same binding pocket of the NTF2 domain prevents phase separation. This can be seen by comparing FIGS. 16A-16C. FIG. 16C illustrates that USP10 can prevent the formation of condensates for G3BP NTF2. Similar competing protein-protein interactions likely play a role in the formation of other biological condensates, e.g. DDX6-dependent P-bodies. Small molecules that disrupt these types of interactions will have a similar effect, i.e. prevent, or in some cases enhance, phase separation.

Referring to FIG. 5, it is seen that this system (350) is similar to the one depicted in FIG. 3, but with the addition of the third fusion protein (300), showing that the second region (125) of the first fusion protein (101) interacts with the first region (315) of the third fusion protein (300). For ease of understanding, these interacting regions are shown graphically as being able to fit together, and also shown with a “+” or “−” symbol. As can be understood, it is expected that the third fusion protein would connect at least two first fusion proteins, allowing the system to connect various self-assembling cores, and thus facilitate large scale phase separation/condensation. An extended connection would require five fusion proteins—a second fusion protein (200) connecting to a first fusion protein (101), connecting to a fixed linker (300), which connects to another first fusion protein (101), which then connects to another second fusion protein (200). In these systems, although it is likely that the connections and interactions between the various fusion proteins does not occur simultaneously, the order of attachment is relatively unimportant.

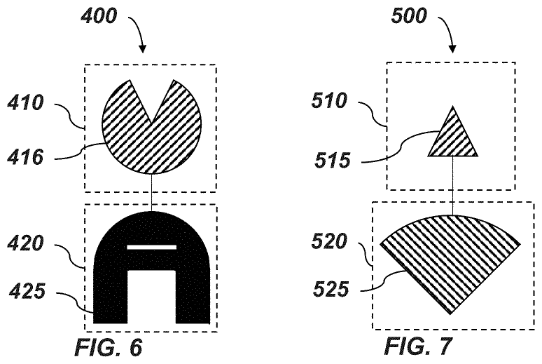

Referring to FIGS. 6-8, a third option is to build on the first option by introducing what can be referred to as a “Protein-Protein Interaction (PPI) Linker”, by including a third and fourth fusion protein (400, 500). This shortens the extended connection seen in the second option from five fusion proteins to four, by incorporating the concept of the “fixed linker” into a variant of the first fusion protein, but the system (450) now requires two different fusion proteins to be introduced instead of just one.

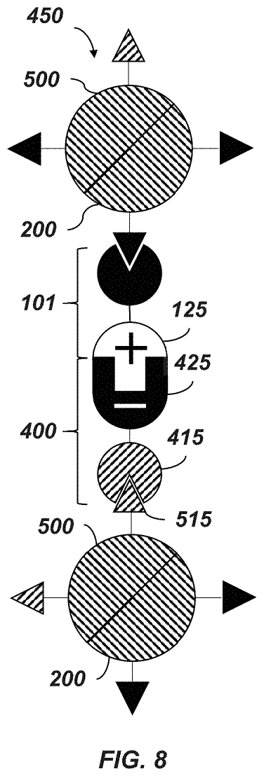

Referring to FIGS. 6 and 8, the third fusion protein (400) can be considered a subset of the first fusion protein (101), and thus is sometimes referred to as an “alternate optoprotein”. The third fusion protein (400) comprises a first region (410) fused to a second region (420), the first region (410) of the third fusion protein (400) comprising a second cognate partner of a second light sensitive protein (415). The second light sensitive protein may or may not be the same light sensitive protein (115) of the first fusion protein/optoprotein. That is, the third fusion protein (400) may or may not be intended to bind to the same light sensitive proteins that the first fusion protein (101) binds to.

The second region (420) of the third fusion protein (400) comprising one or more folded RNA binding domains (RBDs), disordered RBDs, folded non-RBD domains, or combination thereof (425). The second region (420) of the third fusion protein (400) is adapted to interact with the second region (120) of the first fusion protein (100). As seen in FIG. 8, the one or more folded RNA binding domains (RBDs), disordered RBDs, folded non-RBD domains, or combination thereof (425) of the third fusion protein (400) interacts with the one or more folded RNA binding domains (RBDs), disordered RBDs, folded non-RBD domains, or combination thereof (125) of the first fusion protein.

Referring to FIGS. 7 and 8, the fourth fusion protein (500) may be considered a subset of the second fusion protein (200), and thus is sometimes referred to as an “alternate core protein”. The fourth fusion protein (500) comprises a first region (510) fused to a second region (520). The first region (510) of the fourth fusion protein (500) comprises the second light sensitive protein (515), to which the cognate partner (415) present in the third fusion protein (400) or “alternate optoprotein” will bind (see, e.g., FIG. 8). The second region (520) of the fourth fusion protein (500) comprises one or more folded RNA binding domains (RBDs), disordered RBDs, folded non-RBD domains, or combination thereof (525), and is adapted to self-assemble, similar to the second fusion protein (200). As seen in FIG. 8, the second (200) and fourth (500) fusion proteins can self-assemble. The self-assembly is shown as heterogeneous (both second and fourth fusion proteins interact and assemble together), but there may be homogenous self-assembly as well.

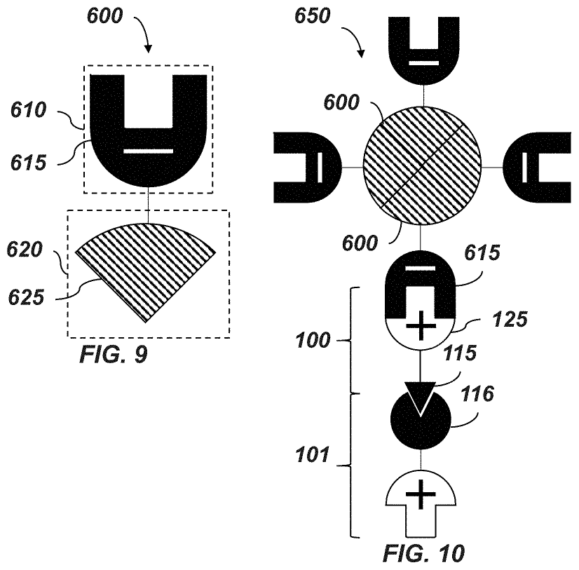

Another alternative option can be seen in reference to FIGS. 9 and 10. This option uses two forms of the first protein to form what can be referred to as an “optolinker”, which can then bind to a modified core protein (a “PPI Core”) via protein-protein interactions. Thus, the protein system (650) consists of at least two first fusion proteins (100, 101). One of the first fusion proteins (100) in the “optolinker” comprises a light sensitive protein (115) fused to one or more folded RNA binding domains (RBDs), disordered RBDs, folded non-RBD domains, or combination thereof (125). The other first fusion protein (101) in the “optolinker” comprises a cognate partner of the light sensitive protein (116) fused to the same one or more folded RNA binding domains (RBDs), disordered RBDs, folded non-RBD domains, or combination thereof (125). When irradiated with the correct wavelengths of light, these two fusion proteins will bind, forming a link that can connect two modified core proteins (600). A system will generally have two or more modified core proteins (600).

The modified core protein (600) is a second fusion protein that comprises first region (610) fused to a second region (620). The first region (610) of the second fusion protein (600) comprises one or more folded RNA binding domains (RBDs), disordered RBDs, folded non-RBD domains, or combination thereof (615). This first region (610) is adapted to interact with the second region (125) of the first fusion proteins. The second region (620) of the second fusion protein (600) comprising one or more folded RNA binding domains (RBDs), disordered RBDs, folded non-RBD domains, or combination thereof (625). This second region (620) is adapted to self-assemble.

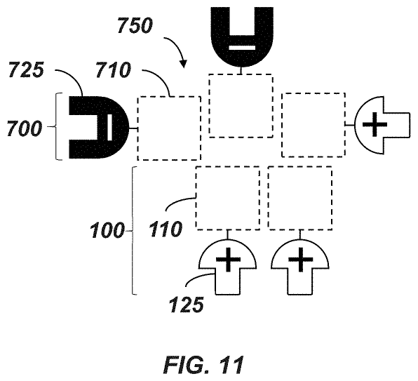

Yet another alternative can be seen in reference to FIG. 11. There, the system (750) can be seen to contain two variants of a first fusion protein. One variant (100) comprises a first region (110) fused to a second region comprising one or more folded RNA binding domains (RBDs), disordered RBDs, folded non-RBD domains, or combination thereof (125), the first region comprising a first light sensitive protein. The second variant (700), or “optoprotein variant”, comprises a first region (710) fused to a second region (720, not shown) comprising one or more folded RNA binding domains (RBDs), disordered RBDs, folded non-RBD domains, or combination thereof (725). The second region comprising one or more folded RNA binding domains (RBDs), disordered RBDs, folded non-RBD domains, or combination thereof (725) is adapted to interact with the second region (120) of the first fusion protein (100) that comprises one or more folded RNA binding domains (RBDs), disordered RBDs, folded non-RBD domains, or combination thereof (125). The first region (710) of the second variant (700) comprises the same first light sensitive protein, and the first regions of both variants (100, 700) are adapted to self-assemble in response to light into an oligomer of at least 2. Thus, one can envision how there could be multiple self-assembled cores connected via the protein-protein interactions between the two variants.

A second aspect of the present disclosure is drawn to a cell line or a stem cell-derived cell that expresses one of the protein systems described above. In some embodiments, the cells may be human cells, yeast cells, cultured neurons, or worm, fly, rodent, or primate models. In some embodiments, one or more genes configured to express the protein system are delivered to the cells utilizing a lentivirus, an adeno-associated virus (AAV), bacterial artificial chromosomes (BAC), transient transfection (e.g. liposomes or proprietary formulations for DNA plasmid introduction), micro-injection, electroporation, or a CRISPR/Cas9-based approach.

A third aspect of the present disclosure is drawn to an expression vector system that comprises at least one expression vector configured to transfect a cell with one or more genes configured to express one of the protein systems described above. In some embodiments, the expression vector system comprises a first plasmid comprising a gene capable of expressing the first fusion protein.

A fourth aspect of the present disclosure is drawn to a method for measuring phase behavior of natural or engineered multi-component condensates.

The method first requires providing one of the protein systems described above. The system may be present inside live cells, or inside or outside dead cells. In some embodiments, the system is present in a well in a multi-well array (plate).

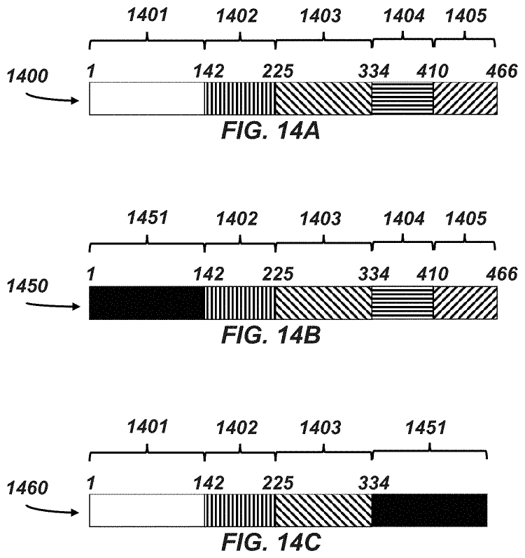

The method then requires oligomerizing the folded RNA binding domain (RBD), disordered RBD, or folded non-RBD domains of the fusion proteins in the protein system by exposing the light-sensitive protein to at least one wavelength of light. For example, when LOV2-SsrA is fused to FTH1, 24-mer ferritin “cores” coated by LOV2-SsrA molecules spontaneously self-assemble. When present with SspB (its cognate partner) and mobile within a cell (that is, in a position to be able to interact with each other), the LOV2-SsrA will eventually attach to the SspB only when irradiated with light having approximately a 450 nm wavelength, but will then detach when not irradiated with such light. By changing the relative concentration of the two components (reference FIG. 18 for calibration methodology used to determine fluorescent protein concentrations in cells), the oligomerization state (valence) can be varied (0 to 24) and intracellular phase diagrams can be quantified, which are amenable to compound- or genetics-based screening applications. Phase separation/condensation generally requires the formation of connected network of interacting biomolecules. The disclosed system and method allow one of skill in the art to engineer constructs that activate phase separation upon light activation, but only if potential protein-protein or protein-RNA interactions are occurring. This in turn allows one to screen for conditions which disrupt said interactions, by finding conditions under phase separation/condensation does not occur, due to the loss of a connected network of interactions. This can be seen in reference to FIGS. 14C and FIGS. 16A-16C.

The system there uses what are referred to as “NTF2 Corelets”. The cores are comprised of a 24-mer ferritin complex coated by iLID molecules (that is, these are similar to the second fusion proteins or “core proteins” described above), which serves as an oligomerization platform mediated by blue light-stimulated sspB-iLID interactions, where the sspB-iLID is fused to the IDR regions and folded NTF2 of G3BP (that is, generally mapping to the first fusion proteins or “optoproteins” described above). By changing the relative concentration of the two species, the oligomerization state can be varied between zero and 24.

The system in FIGS. 16A-16C utilize NTF2 Corelet-expressing U2OS cells. A control can be seen in FIG. 16A, where the NTF2 Corelets are not co-expressed with any other protein of interest. When NTF2 Corelets are co-expressed with CAPRIN10-miRFP670 (see FIG. 16B), an NTF2-interacting protein that preserves its network of protein- and RNA-interactions, the phase boundary is similar. However, when NTF2 Corelets are co-expressed with USP10-miRFP670 (see FIG. 16C), a protein that lacks additional protein-protein interacting and RNA-binding capabilities and thus disengages its network of interactions, no condensates are formed. That is, unlike CAPRIN1, USP10 blocks phase separation of NTF2 Corelets.

In some embodiments, the oligomerization drives gelation of a cytoplasmic ribonucleoprotein (RNP) granule.

The method then requires measuring phase behavior. This can be done by mapping a phase diagram (which may consist of mapping a phase boundary), determining if phase separation, condensation, or aggregation occurs, measuring a condensate material property, a protein concentration, a valence, or some combination thereof.

In some embodiments, the method may also involve providing one or more chemical or biological agents to the well. In some embodiments, the method may also involves utilizing a genetic screen based on gene knockdown (e.g., CRISPR KO, CRISPRi, siRNA, shRNA, or antisense oligonucletides) or gene upregulation (e.g., CRISPRa or DNA plasmid-based overexpression).

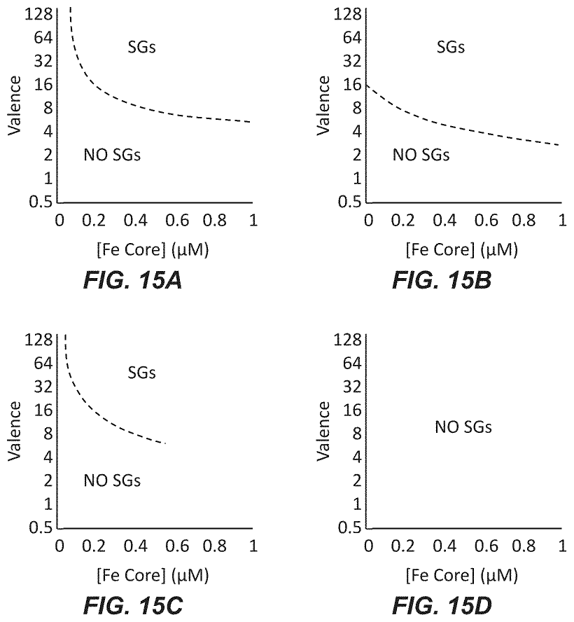

In preferred embodiments, the method further includes determining the impact a genetic screen based on gene knockdown, a genetic screen based on upregulation, the addition of one or more chemical agents to a well, or a combination thereof has, based on the measured phase behavior. That is, using known screening techniques, determine an impact based on the changes in phase behavior. For example, USP10 expression prevents condensation of light-induced G3BP NTF2 oligomers, by disengaging essential protein-protein interaction networks; compounds that target this binding pocket would act similarly. Similar compound-based approaches that disrupt homotypic and heterotypic oligomerization of similar condensate-associated proteins (e.g., NPM1, DCP1A, HSF1, etc.) are possible. Alternatively, compounds that prevent essential RBD-RNA interactions for condensation could be identified (reference FIG. 15D).

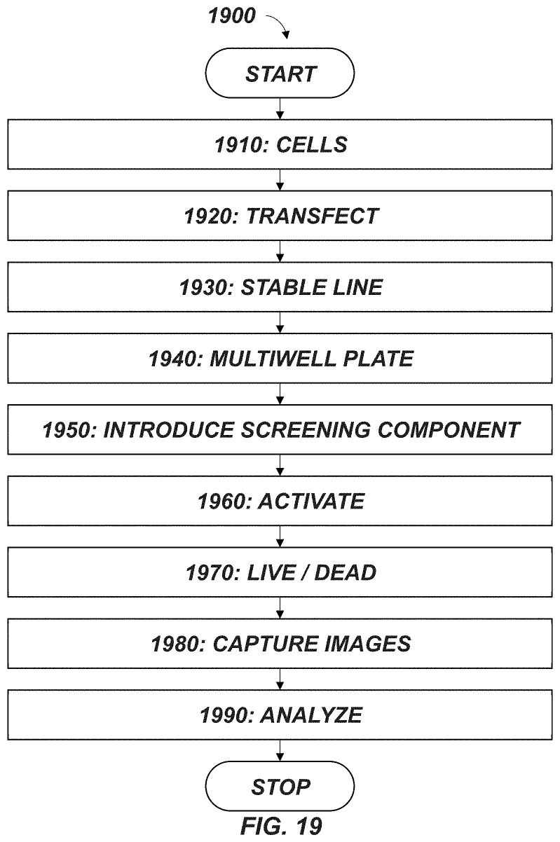

An embodiment of a method can be seen in reference to FIG. 19. There, the method (1900) begins by providing appropriate cells (1910). These cells are then transfected (1920) via plasmids, lentivirus, etc., with the appropriate fusion proteins for the desired system, leading to a stable cell line (1930).

Cells from the stable cell line can be introduced to a multi-well plate (such as a 96- or 384-well plate) (1940).

At this time, one or more screening components may then be introduced (1950) to one or more wells. The screening components may be chemical screening components (e.g., a compound from a small molecule library), genetic screening components (e.g., knockdown, knockout, overexpression, etc.), or some combination thereof. Typically, at this time, there is some delay, during which the screening component is allowed to interact with the cells in well(s).

Then, one or more of the wells are activated by illuminating them with an appropriate wavelength of light, based on the particular light sensitive proteins utilized in the system. This activation can last for any length of time, but preferably occurs for 30 minutes or less, more preferably for 20 minutes or less, and still more preferably for 10 minutes or less. Illumination can be delivered using a laser, light-emitting diode (LED) array, LED lamp, or any such methodology for generating the appropriate wavelength of light.

If the user wishes to fix cells, using any appropriate known fixation technique (e.g., paraformaldehyde, methanol, ethanol, etc.), the cells can be fixed (1970) following activation.

With the live or dead cells, the user may then capture images of the cells (1980), via known microscopy techniques (e.g., confocal, wide-field, super-resolution, etc.). In some embodiments, these images may be captured while sorting live cells, for example using commercially-available equipment known to those of skill in the art, which combines fluorescence activated cell sorting (FACS) with rapid microscopic imaging.

Once the images have been captured, the images may be analyzed (1990). This may involve, for example, determining a degree of condensation, determining a concentration (e.g., by comparing measured intensities to a calibration curve, reference FIG. 18), or determining phase boundaries.

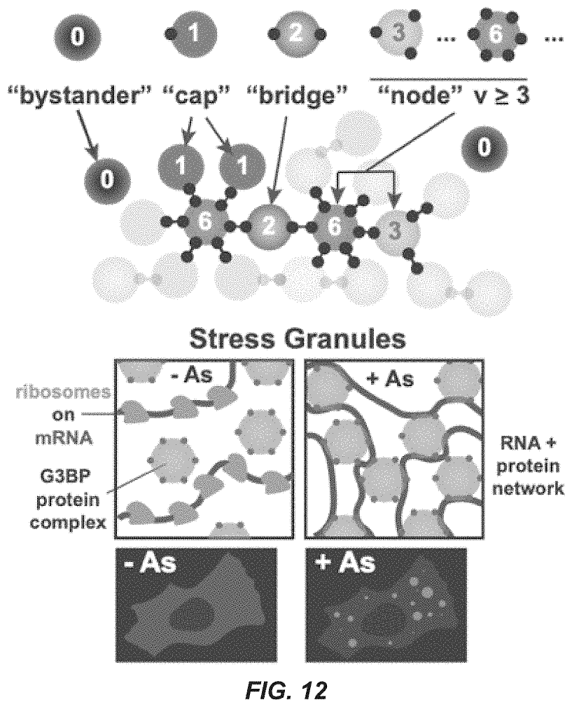

From theory and experiments with patchy colloids (Bianchi et al., 2011) it is known that for a system of interacting particles to phase separate into a dynamically connected network, each particle must have a sufficient number of sites by which it can engage other particles, which defines its valence, v (FIG. 1M); here, the “particle” (or “vertex” in graph theory) could represent an individual protein or RNA molecule, or a stable biomolecular complex. Generally speaking, v>2 is required for phase separation, with higher valences more readily driving phase separation. In the case of a synthetically fused G3BP dimeric complex, there are only two possible interaction interfaces, conferred by the two exposed RBDs, and thus the synthetic G3BP dimer has an overall valence of 2 (i.e. 2 RBD-RNA interfaces); we refer to v=2 particles as “bridges”, which can link different parts of a network, but cannot on their own form a space-spanning interaction network (see FIG. 12)

Given that G3BP's NTF2 domain cannot be replaced by a generic dimerization domain, it can be reasoned that rather than representing a bridge (v=2), the G3BP dimer must somehow embody a particle of v≥3. Objects with v≥3 are referred to as “nodes” (see FIG. 12). In the case of a native G3BP dimer, such valence would be achieved by at least one heterotypic protein-protein interaction (PPI) with the NTF2 domains, in addition to the two RBDs. If so, NTF2 might serve as an interaction platform to link to additional nodes to amplify the valence required for SG condensation.

To screen for SG proteins that might add to overall valence of a G3BP-based complex, one can take advantage of NTF2's dimerization abilities, by enhancing the system via cores. It was hypothesized that NTF2 dimers would create stable homotypic bridges (cross-links) between cores, and heterotypic NTF2-interacting bridges/nodes would partition and confer growth by multiplying valence, allowing identification of such proteins by microscopy.

In a panel of GFP-tagged SG marker proteins (N=20) and P-body proteins (N=3), only 8 SG proteins (USP10, UBAP2L, CAPRIN1, FMR1, FXR1, NUFIP2, G3BP1, G3BP2A) localized strongly to light-induced G3BP ΔRBD Corelets. These proteins are specific to NTF2 interactions, as condensates formed from a self-associating IDR from another protein, FUS, only recruit FUS ΔNLS.

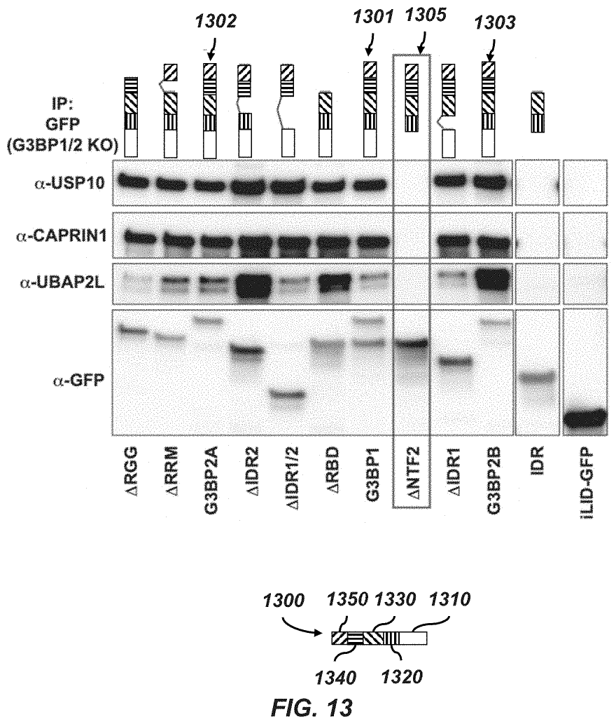

Consistent with the results of the Corelet NTF2 interaction assay, G3BP-mediated coimmunoprecipitation (co-IP) of USP10, CAPRIN1, and UBAP2L all require its NTF2 domain, and interactions are preserved following RNase treatment and stringent wash steps (FIG. 13).

These identified proteins could serve as G3BP-interacting bridges or nodes to contribute to the overall valence requirements for SG condensation, given that all but USP10 feature an RBD. Knockout of USP10, CAPRIN1, NUFIP2, FXR1/FXR2/FMR1 (3KO), or FXR1/FXR2/FMR1/NUFIP2 (4KO) had no effect SG formation, suggesting these components play a limited role in overall valence amplification. Moreover, the arsenite-triggered phase threshold was not significantly modulated by endogenous levels of USP10 or CAPRIN1, as triple-KOs (G3BP1/G3BP2/USP10, G3BP1/G3BP2/CAPRIN1) do not require substantially different amounts of G3BP for rescue relative to G3BP1/2 double-KO. By contrast, UBAP2/2L double-KO cells display SGs of reduced size, which form in only a minority of cells; these data suggest the possibility that UBAP2L might act as an additional critical node. Support for this comes from the serendipitous discovery of a missense mutation in G3BP (S38F), which were found to abrogate its ability to rescue stress granule formation in G3BP KO cells, even when expressed at levels >10-fold higher than the WT G3BP rescue threshold. G3BP S38F retains homo-dimerization and USP10-binding capacity and partitions strongly into stress granules formed by WT G3BP. However, it is unable to form high-affinity complexes with CAPRIN1 and UBAP2L, suggesting that the mutation changes G3BP from a v≥3 node to a v=2 bridge, which is no longer able to bring in additional overall valence from UBAP2L. Conversely, a previously described G3BP F33W variant that is unable to bind USP10 or CAPRIN1 but rescues SG formation, retains its node identity via association with UBAP2L and displays a similar threshold concentration for rescue as WT. Taken together with the findings above, these data provide strong evidence that G3BP dimers must serve as nodes to engage UBAP2L, in order to fulfill their essential role in SG condensation.

High valence G3BP RBD nodes are sufficient for stress granule formation with attached P-bodies

The data suggests that RBD complexes of sufficiently high valence are likely required for stress granule formation, but a stringent test of this model and determination of the minimal valence for condensation required a synthetic intracellular reconstitution approach. To quantitatively interrogate the relationship between RBD valence and the threshold protein complex concentration for stress granule formation, the Corelet system described previously (the cores are comprised of a 24-mer ferritin complex coated by iLID molecules, which serves as an oligomerization platform mediated by blue light-stimulated sspB-iLID interactions, where the sspB-iLID is fused to the IDR regions and RBDs of G3BP) can be utilized to quantitatively map valence and concentration-dependent phase diagrams.

FIG. 14A shows five domains of interest in G3BP (1400): Replacing the valence-amplifying dimerization domain (NTF2) of G3BP with a synthetic valence-amplifying sspB node (FIGS. 14A, 14B), it is found that non-stressed cells require a very high degree of oligomerization (valence ˜24 at 0.15 μM Core) to drive condensation (See FIG. 15A). However, upon arsenite treatment, condensation occurs at much lower concentrations and valencies (valence ˜8 at 0.15 μM Core) (See FIG. 15B), and the resulting granules are significantly larger, relative to non-stressed cells. This valence-dependent phase separation occurs rapidly (within seconds) and is fully reversible regardless of activation time (5 vs. 60 min), indicating that multivalent RNA-binding contacts are essential for both formation and maintenance of stress granules. Moreover, it is found that these condensates mimic the properties of endogenous SGs, including a dependence on influx of exposed RNA), recruitment of canonical SG proteins and polyadenylated mRNA, attachment of P-bodies, and liquid-like fusion with dynamic rearrangement of internal components. These structures are referred to as opto-SGs (optogenetic stress granules).

The shift in the ΔNTF2 Corelet opto-SG phase threshold after treatment with arsenite can be dramatically visualized in cells exposed to successive 5-minute cycles of blue light-activation and de-activation. Arsenite treatment triggers de novo assembly of opto-SGs in a time- and valence-dependent manner, with assembled opto-SG becoming progressively larger on a timescale component consistent with SG assembly in WT cells. The arsenite-driven shift in the opto-SG phase diagram is negated by pre-treatment with cycloheximide, which blocks disassembly of polysomes following translational arrest (See FIG. 15C). Moreover, long-term inhibition of RNA production induced by Actinomycin D prevents the formation of opto-SGs (See FIG. 15D). These drug-dependent changes in opto-SG assembly are not artifacts of the Corelet system, as similar shifts in the phase threshold are absent in the case of a control self-associating IDR (FUS IDR) that lacks ability to bind RNA.

These data show that the formation of light-triggered opto-SGs is greatly enhanced by polysome disassembly, which is expected to flood the surrounding cytoplasm with RNA that would act as a nucleic acid-based node with very high valence (i.e. binding sites or bridges for RNA-binding proteins). To examine which G3BP domains are essential for opto-SG condensation, iterative truncations of G3BP ΔNTF2 were fused to sspB and examined their response to light in the Corelet system. Consistent with a lack of partitioning into SGs, G3BP's disordered linker is unlikely to engage in significant self-interactions, as G3BP IDR1, IDR2, and IDR1/2 Corelets never cause phase separation, irrespective of drug treatment. In contrast, polyA+ opto-SGs containing all tested SG markers are assembled by Corelets containing IDR2-RBD (RRM and RGG) or just the RBD. Moreover, the Corelet system recapitulates critical features associated with expression of GFP-tagged truncated variants.

First, ΔNTF2/ΔIDR2 (i.e. analog of GFP-G3BP1 ΔIDR2 that effectively lack RNA-binding capacity due to local electrostatic repulsion) fails to form granules in all conditions tested. Second, ΔNTF2/ΔIDR1 forms more irregular granules, similar to GFP-ΔIDR1. Third, the phase threshold for RBD-only Corelets is right shifted relative to ΔNTF2 (i.e. containing IDR1/2), consistent with the higher concentration of GFP-tagged ΔIDR1/2 expression required for rescue. Fourth, relative to ΔNTF2 Corelets, all ΔNTF2/ΔIDR1 Corelets recruit SG proteins and polyA+ RNA similarly, and exhibit enhanced and reversible phase separation upon successive light-dark cycles following arsenite treatment. Finally, like endogenous stress granules, all G3BP opto-SGs form multiphase structures with DDX6-positive P-bodies; importantly, this suggests that, in each case, the high valence G3BP Corelets confer sufficiently unfavorable interactions with the P-body interaction network to give rise to phase immiscibility. However, unlike GFP-tagged G3BP variants, opto-SG formation requires both the RRM and RGG segments of the RBD, which could reflect steric hindrance of a closely juxtaposed core.

Stress granules with attached P-bodies are the default condensate associated with high valence RBD nodes

High valence G3BP RBD nodes (but not dimeric bridges) are sufficient to induce stress granule formation following polysome disassembly, but it is unclear whether this is a unique feature of G3BP or common to RNA-binding nodes that interact with G3BP NTF2. We reasoned that if such NTF2-associated RBPs contribute essential, additive RNA-binding valence to the multi-protein complex, synthetic high valence nodes connected to individual RBDs would nucleate opto-SGs in isolation (i.e. mimic G3BP RBD Corelets). To test this, the RBDs of CAPRIN1 and UBAP2L were oligomerized utilizing the Corelet system, and mapped phase diagrams in both non-treated and arsenite-exposed cells. Surprisingly, despite each containing only a single RGG region, their phase thresholds are shifted toward lower concentrations and Corelet valence relative to G3BP RBD (1 RRM and 1 RGG), indicating an enhanced propensity to drive mRNP condensation; these results are not artifacts of the Corelet system, as SG rescue experiments using GFP-tagged chimeric G3BP proteins show that such RBDs can substitute for G3BP RBD, with a similar enhanced propensity. Moreover, in each case, arsenite-induced polysome disassembly causes a shift in the phase threshold as well as the growth of reversible polyA+ opto-SGs, which are positive for a panel of SG 15 markers. Again, each of these RBD Corelet-mediated opto-SGs are attached to P-bodies, suggesting that the CAPRIN1 and UBAP2L RBDs are alone sufficient to confer immiscibility with the P-body phase. It is noted that the arsenite-induced shift in phase threshold is relatively small compared to G3BP RBD, which may suggest that stress has differential effects on the ability of distinct RBDs to bind disassembled polysome substrates relative to intact polysomes, or that specific RBDs may feature intrinsic self-interactions that contribute to phase separation. The latter possibility was ruled out for the aromatic-rich CAPRIN1 RGG, as RNA depletion (Actinomycin D) abrogates phase separation. Moreover, RGG-mediated condensation is not simply due to net positive charge conferred by high abundance of arginine residues, as scrambling CAPRIN1's RGG region prevents phase separation. The RBD (2 KH and 1 RGG) of FXR1, a dimeric RBP that stably associates with UBAP2L, is also capable of assembling stress-regulated, reversible, polyA+ opto-SGs with expected SG markers and attached P-bodies. Based on a large panel of RBDs placed in the Corelet system, synthetic nodes with high RBD valence are sufficient to nucleate polyA+ SGs, irrespective of whether they are associated with a SG or P-body protein or linked to G3BP IDR. Nevertheless, different Corelets can plug into alternative condensate-forming interaction networks, as full-length P-body protein (DCP1A) Corelets that retain the ability to engage in PPI bridges with P-body proteins recruit a panel of additional P-body markers but not SG proteins. Further, condensate protein composition, P-body attachment, and relative phase thresholds are not determined by the type or number of RNA-binding motifs. Thus, polyA+, SG-like condensates coupled to immiscible P-bodies are the “default” option for multivalent RBD nodes, and condensate specificity is likely dictated by the network connectivity of particular protein-protein interaction nodes.

Competition between protein-protein interaction nodes encodes multiphase condensation.

As synthetic high valence versions of their associated RBDs are sufficient to cause SGs, UBAP2L/FXR or CAPRIN 1/FXR complexes might compensate for G3BP KO if individual proteins could mimic G3BP nodes (e.g., network centrality) and are expressed at similar levels. Unlike G3BP, CAPRIN1 does not rescue, even at relatively high levels, suggesting that it acts primarily as an RNA-binding bridge in the SG network. Conversely, mild overexpression (<1 μM) of UBAP2L or FXR1 is sufficient for the formation of polyA+ SGs in the absence of G3BP, suggesting that the two proteins act as SG nodes that engage sufficient RBD network valence for SG condensation. Inspired by the case of G3BP, it was hypothesized that a self-association domain would confer the requisite valence for node identity (v≥3). Although previous studies have indicated that such a domain (dimerization) exists for FXR1, a self-associating interface for UBAP2L has yet to be identified.

A fragment scanning Corelet screen was performed for UBAP2L regions with FUS IDR- (weak self-association) or NTF2-like (dimerization) properties, using fragments from CAPRIN1 (predicted bridge) as an internal control. Of 13 fragments tested, UBAP2L 781-1087 was unique in forming stress-independent, polyA-negative droplets, properties which were conserved upon further truncation of this aromatic-containing, FUS-like region. The predictive power of the Corelet domain screening approach is apparent in that the identified C-terminus is essential for UBAP2L's role in G3BP-independent SG formation (i.e. deletion turns UBAP2L from a node into a bridge). UBAP2L does not form high affinity complexes with its ortholog UBAP2, yet the protein is highly conserved, including the identified self-association domain. Considering this in light of the UBAP2/2L double KO phenotype, it is surmised that the region forms weak self-associations between UBAP2/2L proteins in separate high-affinity complexes (e.g. FXR1/UBAP2L, UBAP2L/G3BP), thus acting as an essential valence multiplier for SG condensation. Since high-affinity UBAP2L complexes containing both FXR1 and G3BP are scarce or non-existent, we hypothesized that G3BP and FXR nodes compete for limiting amounts of the connecting node UBAP2L and that relative stoichiometry is critical for their mixed distribution in endogenous SGs. Consistent with this, ectopic over-expression of FXR1 causes multiphase SGs, which is likely at play in the endogenous system, as STED microscopy on G3BP1 in live SGs shows micro-heterogeneity that was not visible with conventional microscopy. Conversely, UBAP2L forms single phase SGs with G3BP. Co-expression of all three proteins reveals that at constant levels of UBAP2L, stoichiometry between FXR1 and G3BP nodes is critical for determining whether single or multi-phase compartments result: high relative FXR1 causes demixing from G3BP with UBAP2L present in both phases; conversely, high G3BP results in a single phase of all three proteins. Thus, competition between non-neighboring nodes (G3BP, FXR1) for a limited supply of a connecting node (UBAP2L) appears to determine the degree to which networks intertwine to form a single miscible phase. Unexpectedly, we observed that both FXR1 and UBAP2L nucleate small, stress independent granules in G3BP KO cells, which fuse and grow after stress.

As cells lack the SG node of greatest centrality, it was hypothesized that these G3BP-independent condensates tie into different PPI networks than endogenous SGs. Indeed, UBAP2L condensates contain both SG and P-body proteins, which may result from UBAP2L's high-affinity association with the essential (Ayache et al., 2015; Ohn et al., 2008) P-body node DDX6. Intriguingly, DDX6 is weakly recruited to SGs, whereas EDC3 and DCP1A are repelled, which reflects relative preferences for one of the two immiscible networks.

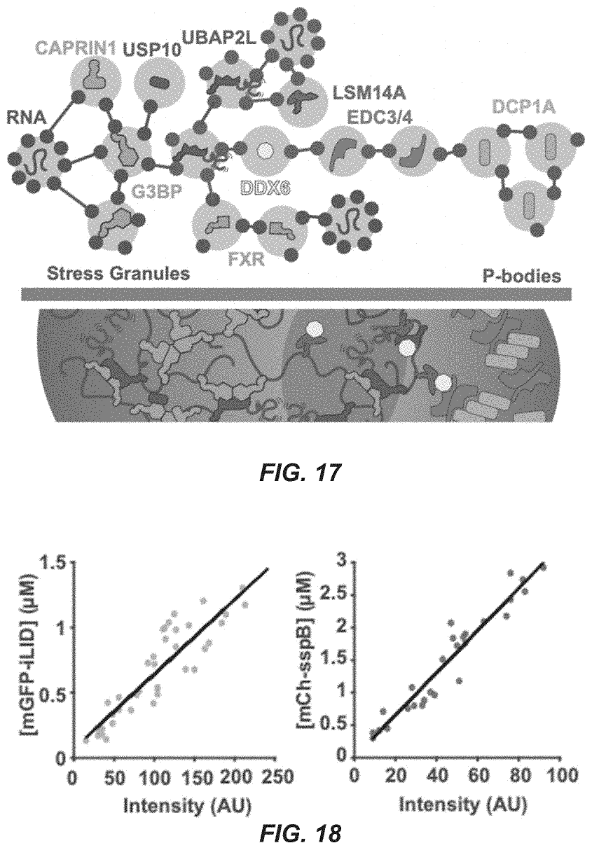

These studies and past work support a continuum of nodes that overlap with respect to connectivity—e.g. G3BP and FXR1 vs. UBAP2L (reference FIG. 17 for a graphical summary of the interaction networks supported by this series of experiments). We hypothesized that such node-based competitions would cause restructuring of the global P-body and SG networks, which would be observable on the scale of phase miscibility.

To test this, central SG and P-body nodes were expressed pairwise in G3BP KO cells, examining multiphase patterning after stress. Depending on network distance between nodes, the two proteins were observed to be miscible, multiphase, or exist in separate condensates. In contrast to neighboring nodes that form single phases (e.g. G3BP and UBAP2L, EDC3 and DCP1A), upregulation of distant nodes (e.g. G3BP and DCP1A) decouple SGs from P-bodies. While such studies suggest that competing PPI networks are sufficient to encode distinct multiphase condensates, the degree to which network-substrate interactions play an auxiliary role is unclear. If PPI networks with unfavorable interactions are the prime mediator of SG/PB demixing, we predict that even disconnected networks that share the same substrate preference would be immiscible. An ideal model to test this are synthetic G3BP/UBAP2L RBD nodes, which encode condensates with identical properties to endogenous SGs, yet lack PPI connectivity through the missing NTF2 domain.

As proof of principle, G3BP RBD opto-SGs form on the surface of stress-induced UBAP2L condensates and retain multiphase properties throughout maturation. Upon deactivation, opto-SGs dissolve, and vanishing of surface tension leads to dispersal of the UBAP2L phase into individual puncta.

Multiphase condensates are similarly observed across a panel of co-expression pairs for G3BP/UBAP2L-associated RBD nodes and their FL counterparts; note in particular how UBAP2L RBD Corelets form striking multiphase condensates with FL UBAP2L, although such multiphase behavior is less clear with G3BP RBD Corelets co-expressed with FL G3BP1, at diffraction-limited scales. As identical experiments with NTF2 Corelets universally result in single-phase structures, it can be concluded that shorter network distance, i.e., direct protein-protein interaction, promotes miscibility while longer network distance, i.e., binding through RNA intermediate, promotes multiphase behavior.

Method Details

Plasmid Construction

Unless indicated (e.g. pHR lentiviral vector, SFFV promoter), all lentiviral DNA plasmids used the FM5 lentiviral vector, which features the Ubiquitin C promoter. DNA fragments encoding our proteins of interest were amplified by PCR with Phusion® High-Fidelity DNA Polymerase (NEB). Oligonucleotides used for PCR were synthesized by IDT. In-Fusion HD cloning kit (Clonetech) was used to insert the PCR amplified fragments into the desired linearized vector, which featured standardized linkers and overlaps to allow cloning in high throughput. Cloning products were confirmed by GENEWIZ sequencing, sequencing from both ends of the insert. For all sspB-mCherry-tagged DNA constructs, correct sequencing was confirmed a second time by an independent researcher. Stress granule (SG) rescue defects associated with the G3BP S38F mutant were confirmed using two different fully sequenced DNA constructs (FM5-mGFP-G3BP1 S38F and pcDNA4 t/o- GFP-G3BP1 S38F) generated by two separate labs.

Cell Culture

Cells were cultured in DMEM (GIBCO) with 10% FBS (Atlanta Biological), supplemented with 1% streptomycin and penicillin, and kept in a humidified incubator at 37° C. and 5% CO2. All cell lines tested mycoplasma-negative. HEK293 and HEK293T cells were kind gifts from Marc Diamond lab (UT Southwestern). HeLa cells were obtained from ATCC. U2OS cells and U2OS G3BP1/2 KO cells were previously described (Kedersha et al., 2016). This knock-out cell line was extensively characterized in the cited paper, and multiple independent labs have validated resistance to stress granule formation (personal communications). G3BP1/2 KO (hereafter, described as G3BP KO) was confirmed internally by Western blot.

Generation of Lentivirus and Lentiviral Transduction.

All live cell imaging experiments were performed using cells stably transduced with lentivirus, with the exception of light-induced sspB-/iLID-ΔNTF2 dimer-mediated rescue of G3BP knockout (see Transient transfection). Lentiviruses containing desired constructs were produced by transfecting the plasmid along with helper plasmids VSVG and PSP (from Marc Diamond lab, UT Southwestern) into HEK293T cells with Lipofectamine™-3000 (Invitrogen). Virus was collected 2-3 days after transfection and used to infect WT U2OS, G3BP KO U2OS, or WT HEK293 cells. Lentivirus transduction was performed in 96-well plates. Three days following lentivirus application to cells at low confluency, cells were passaged for stable maintenance or directly to 96-well fibronectin-coated glass bottom dishes for live cell microscopy. For non-Corelet experiments, stable cells were passaged at least 3 times over 8+ days prior to use in live cell imaging experiments to eliminate cells expressing lethal levels of the fusion protein of interest. In all experiments, 90%+ of cells featured expression of the protein of interest at a range of concentrations (typically <5 μM; estimated concentrations are noted as relevant). This specific protocol was designed to avoid artifact-prone concentrations of fusion proteins that can occur with lipid-based transient transfection.

Transient Transfection

Unlike other experiments (see above), light-induced ΔNTF2 dimer-mediated rescue of G3BP knockout was performed using transient transfection. Initial attempts to rescue defects (data not shown) using lentivirus-based transduction were not successful due to inability to reach high concentrations of the individual fusion proteins (i.e. >5 μM of both mCherry-sspB-G3BP ΔNTF2 and mGFP-iLID-G3BP ΔNTF2). Thus, individual wells of a 96-well plate containing G3BP1/2 KO U2OS cells were transfected with both mCherry-sspB-G3BP ΔNTF2 and mGFP-iLID-G3BP ΔNTF2 using Lipofectamine™-3000 (Invitrogen) according to manufacturer's recommendations. 24-hours later, cells were observed to feature both fusion proteins diffusely expressed throughout the cytoplasm. Arsenite was added to a final concentration of 400 μM. 1-hour later, cells were imaged. Three biological replicates were performed. In rare cells with very high concentrations of both components (>10 μM of each), stress granules were observed, regardless of time of blue light activation. The light-independent nature of dimer-based rescue at these concentrations is consistent with the measured in vitro dark state K_{d} of 4.3 μM for iLID-sspB (Guntas et al., 2015). At such concentrations, iLID and sspB are expected to associate strongly in the dark. The in vitro light state K_{d} for iLID-sspB is 0.2 μM (or ˜10 nM for “core” measurements, see Phase diagram data collection), which sets the lower limit for the assay.

Live Cell Confocal Microscopy

Cells were imaged on fibronectin-coated 96-well glass bottom dishes (Cellvis). Confocal images were taking on a Nikon A1 laser scanning confocal microscope using a 60× oil immersion lens with a numerical aperture of 1.4. The microscope stage was equipped with an incubator to keep cells at 37° C. and 5% CO₂. Proteins tagged with mCherry, mGFP (GFP), EYFP, and miRFP670 (iRFP) were imaged with 560, 488, 488, and 640 nm lasers, respectively. The above details apply to all imaging data in the manuscript was the exception of STED super-resolution and widefield microscopy images. See below for details.

Stimulated Emission Depletion (STED) Super Resolution Microscopy

For single channel STED images, sequential image sets (each line imaged concurrently with and without the STED laser to control for bleaching artifacts) were taken with increasing STED power using the ‘Custom Axis’ options available in Imspector. For dual channel STED images, two sequential image sets were taken with each line imaging mGFP (+/−STED) and miRFP (+/−STED) with the first mGFP STED power set to 0% STED power to avoid miRFP image bleaching, which occurred during the second image (again using the ‘custom axis’ option available in Imspector).

Widefield Microscopy

For some images, G3BP KO or UBAP2L KO U2OS cells stably expressing GFP-UBAP2L were grown on glass coverslips, stressed with 400 μM arsenite when indicated, and fixed using 4% paraformaldehyde in PBS for 15 minutes, followed by 5 minutes post-fixation/permeabilization in ice cold methanol. Cells were blocked in 5% horse serum/PBS, and primary and secondary incubations performed in blocking buffer for 1 hour with rocking. Following washes with PBS, cells were mounted in polyvinyl mounting media and viewed. Images were captured using a Nikon Eclipse E800 microscope with a 63× Plan Apo objective lens (NA 1.4) and illuminated with a mercury lamp and standard filters for DAPI (UV-2A 360/40; 420/LP), Cy2 (FITC HQ 480/40; 535/50), Cy3 (Cy 3HQ 545/30; 610/75), and Cy5 (Cy 5 HQ 620/60; 700/75). Images were captured using a SPOT Pursuit digital Camera (Diagnostics Instruments) with the manufacturer's software, and raw TIF files were imported into Adobe Photoshop CS3. Identical adjustments in brightness and contrast were applied to all images in a given experiment.

Corelet Activation

Pre-activation and post-activation images of G3BP KO cells stably expressing the indicated fusion proteins were captured with the mCherry (560) channel only to visualize the sspB component without triggering light-induced dimerization with the iLID-mGFP tagged Ferritin core. Cells were activated with a 488-laser using 1% laser power to cause dimerization of iLID and sspB. Activation of cells was achieved by imaging the mCherry and mGFP channels simultaneously using a 6-second frame interval for an area of 120×120 μm2 (1024×1024 pixels) at Nyquist zoom. See also Phase diagram data collection.

Fluorescence Recovery After Photobleaching (FRAP)

G3BP KO cells stably expressing indication fusion proteins were first globally activated (i.e. iLID-sspB dimerization) by constantly exposing them with the 488 laser for 5-minutes. Light-activated condensates were then bleached in a ˜1 μm² region with the 560 laser at high power to quench the majority of the mCherry-sspB component of the condensate. Fluorescence recovery was monitored while imaging both mCherry and mGFP channels at a frame interval of 6-seconds. Fluorescence was standardized based on a non-FRAPed droplet to control for bleaching and fluorescence intensity was compared to the initial image for plotting purposes. Cell treatment with arsenite to dissociate polysomes Cells were treated by adding sodium arsenite to cell media at a concentration of 400 μM, which is in excess of saturating concentrations for polysome disassembly (Kedersha et al., 2016). Images were captured between 50-minutes and 2-hours (typically 1-hour) after arsenite treatment, unless performing light-dark cycling experiments (see below). No differences were observed with respect to rescue, phase threshold shift, SG inhibition, etc. between 60- and 120-minutes. SG number/size typically peaked by 45-minutes, and 1- to 2-hour time window was chosen, so drug reached maximal effect. Cells typically began to die ˜6 hours following treatment, so to avoid toxicity/lethality confounds, the indicated 1- to 2-hour time window was used. Inhibition of polysome disassembly by pre-treating with cycloheximide was added to a final concentration of 100 μg/mL (G3BP KO cells expressing indicated fluorescent fusion proteins). Following 30-minutes of incubation, arsenite was added at a concentration of 400 μM. 1-hour later, cells were assessed for formation of stress granules (GFP-G3BP rescue) or activation cycles were performed (Corelets).

Cell Treatment with Actinomycin D to Inhibit Transcription

Actinomycin D dissolved in DMSO was used to treat G3BP KO cells expressing indicated Corelets at a concentration of 5 μg/mL. Images were taken 12-18 hours after actinomycin D treatment, a time interval during which nucleoli were no longer apparent by bright field observation and the vast majority of mRNA was expected to be degraded. Final concentration of DMSO was 0.5%. For Actinomycin D plus arsenite experiments, arsenite was added at a concentration of 400 μM˜12 hours following actinomycin D treatment and cells were imaged 1-2 hours subsequently. Qualitative observations suggested that the application of Actinomycin D at the indicated concentration was lethal following ˜30-36 hours of treatment. Time point was chosen to maximize the time since treatment (i.e. to reduce RNA cells by as much as possible) without extensive lethality from the drug.

Phase Diagram Data Collection

In order to determine precise phase threshold boundaries for intracellular phase diagrams, analyzed cells must feature high variability with respect to sspB-mCherry and iLID-mGFP stoichiometries to sample sufficient core concentrations and valencies. In order to achieve a broad concentration range for both components, G3BP KO cells were transduced in 96-well plates (Cellvis) using an arrayed lentivirus approach. In this protocol, rows varied from 2 to 60 μL iLID-GFP-Fe lentivirus; columns, 2 to 60 μL mCherry-sspB-open reading frame (ORF)/ORF-mCherry-sspB lentivirus. G3BP KO cells were plated directly into the arrayed lentivirus to attain ˜25% confluency upon subsequent attachment to the plastic substrate. 72-hours later, at confluency, all 16 wells associated with an individual Corelet condition were washed with PBS, trypsinized, quenched with fresh media, and combined, thus ensuring a diverse population of cells with highly variable iLID to sspB ratios. Cells were plated at a 1:8 dilution factor onto fibronectin-coated, glass bottom 96-well plates (Cellvis) and imaged 48 hours later at 60-90% confluency.

For all data collected toward generation of phase diagrams, a standardized imaging protocol was used to avoid confounds related to alterations in microscopy settings. Identical imaging settings were used relative to fluorescence correlation spectroscopy (FCS)-based calibrations (fluorescence to absolute concentration) (see Quantification and Statistical Analysis). Specifically, images were collected using 0.5 frames per second scan rate, 1024×1024 pixel frame, and 1.75× Nyquist zoom (63× oil immersion lens). Laser powers (1% 488 and 100% 546), intensities, and gains were kept constant. All time lapses were 5-minutes in length and featured 6-second intervals between frame acquisition. Following the last frame, laser intensity was dropped for 4 additional frames followed by acquisition of 4 final images at higher relative laser intensity.

This protocol was selected to achieve wide dynamic range (e.g. to achieve sufficient resolution of lower concentration cells, which feature higher signal to noise) and to avoid pixel saturation in cases of Corelets that cause dense, exceptionally bright puncta. Using this standardized protocol, each 5-minute acquisition was able to add (on average) 10 data points to the phase diagram. Thus, an average phase diagram used in this study required collection of 20-30 fields or ˜2 hours of data acquisition time. Typically, an individual phase diagram was compiled from data collected over the course of 3-5 experiments (i.e. different lentivirus transductions on different days). However, certain phase diagrams featured data from significantly more experiments (e.g. G3BP ΔNTF2 Corelets, a condition used as a positive control for effect of drug treatments throughout studies, which ensured quality control).

Throughout the duration of the study, there was no indication of systematic changes with respect to drug response, drug efficacy, or measurement of fluorescence intensities. When selecting cells for analysis, only fully activated cells (i.e. entire cell was within field of view) were considered to avoid potential artifacts related to local activation and diffusive capture (Bracha et al., 2018). The average mCherry and mGFP fluorescence intensity for a cell was determined using the first frame (i.e. prior to blue-light mediated dimerization of iLID on core to sspB-tagged protein of interest), and manual image segmentation of 4.5×4.5 μm square regions of interest (ROIs) in cytoplasmic regions featuring homogenous fluorescence (i.e. regions with low density of membrane-bound organelles like the endoplasmic reticulum). The aforementioned FCS calibration curves (FIG. 18) were then used to determine the mCherry and mGFP concentrations. The mGFP concentration was divided by 24 (subunits per ferritin complex or “core”) to determine the core concentration. Valence was determined for an individual cell by dividing the mCherry value by the core value.

This is a highly accurate measure based on the lever rule—in a “one-component” system (i.e. FUS IDR Corelets, which feature minimal endogenous proteins, nucleic acids) consistency in valence between initial, dilute, and condensed phases is reliably observed. Binary decisions regarding Corelet-mediated phase separation in a cell of interest were determined manually. Datasets used for subsequent automated generation of phase diagrams and phase diagrams were coded and sent to a separate individual.

Cycling Experiments Following Drug Treatments

Cycling experiments were performed as described in Phase diagram data collection with minor changes. After treatment of G3BP KO cells expressing indicated sspB/iLID Corelets with arsenite (or indicated drug), image acquisition was immediate commenced. For most experiments, a 5-minute activation time lapse was acquired for each cycle, immediately followed by a 5-minute time lapse for deactivation. We have determined that this deactivation time far exceeds that which is required for complete reversibility (i.e. typically 30-60 seconds), based on studies of diverse proteins in the Corelet system. Indicated cycling parameters were repeated 6-8 times. In certain experiments, instead, 10-minute activation time lapses were immediately followed by 5-minute time lapse for deactivation. This was repeated four times. Intervals were kept constant at 6-seconds. Representative cells/fields were chosen for data analysis based on standard core concentrations (0.25 μM) and desired valence.

G3BP Rescue Competition, and Stress Granule Inhibition Experiments

For G3BP rescue competition experiments, an identical arrayed lentivirus approach was used as described in Phase diagram data collection (i.e. 2-60 μL G3BPmCherry and 2-60 μL mGFP-ORF, arrayed 4-wells by 4-wells for 16-wells total of a 96-well plate). G3BP KO cells were plated into lentivirus then combined and passaged 72-hours later.

Live cell confocal microscopy was performed on Day 5 post-transduction. For each condition (GFP-tagged protein of interest), 4 separate experiments (each experiment=1-well/arsenite treatment) were performed on three separate days with numerous technical replicates (fields of view). Live confocal imaging was performed 1-2 hours following arsenite treatment. mCherry and mGFP concentrations were determined similarly as for phase diagrams and manual scoring of stress granule absence or presence was performed. Similar protocols were used to assess rescue in the absence of competition.

For stress granule inhibition experiments, WT U2OS cells stably expressing YBX1-mCherry (SG marker) were plated into 96-well plates at 25% confluency and transduced in arrayed format with 2-60 μL lentivirus of indicated mGFP-tagged protein. Three days later, cells were washed, trypsinized, combined, and passaged. Three days after this, cells were passaged onto fibronectin-coated 96-well plates. Live cell confocal imaging was performed 2-days later (i.e. 8-days following lentivirus transduction) when cells were at 60-80% confluency. Images were taken between 1-2 hours after arsenite treatment. 3-4 independent experiments were performed for each condition on two separate days with numerous technical replicates (i.e. fields of view) per experiment. Concentrations of mGFP-tagged protein was determined, SG formation was assessed in a binary manner, and all data was coded then sent to a separate individual for quantitative analysis.

Stress Granule Partitioning

For stress granule partitioning experiments, WT U2OS cells stably expressing mGFP-CAPRIN1 or mCherry-CAPRIN1 were plated into 96-well plates at 25% confluency and transduced with either 30 μL of indicated mCherry-tagged lentivirus (mGFP-CAPRIN1) or mGFP-tagged lentivirus (mCherry-CAPRIN1 cells). Three days later, cells were washed, trypsinized, binned, and passaged onto fibronectin-coated 96-well plates. Imaging was performed 2-days later when cells were at 60-80% confluency. Images were taken between 1-2 hours after arsenite treatment. 3 independent experiments were performed for each condition.

Co-localization Corelet studies followed similar protocol as “Phase diagram data collection” but performed using two lentivirus co-transduction (with non-fluorescence iLID-Fe instead of typical GFP-tagged version) on G3BP KO cells stably expressing the indicated GFP-tagged protein. 72-hours after infection, passaged at 1:8 dilution factor onto fibronectin-coated, glass bottom 96-well plates (Cellvis). 48-hours later, treated with arsenite (400 μM). One hour later, removed plate from humidified incubator and placed on a blue LED light illuminator (Invitrogen SafeImager 2.0) for 10-minutes to activate Corelets. Immediately fixed with 4-percent PFA for 10-minutes. Washed twice with PBS and permeabilized with ice cold 70% methanol for 10-minutes. Washed an additional two times with PBS then placed at 4° C. overnight. Performed fixed cell confocal microscopy the next day to examine colocalization of opto-SGs with indicated GFP-tagged SG/PB proteins.

RNA Fluorescence In Situ Histochemistry

Fixed indicated cells with 4-percent PFA for 10-minutes. Washed twice with PBS, permeabilized with ice cold 70% ethanol, and placed at −4° C. overnight. Replaced ethanol with Wash Buffer A (Stellaris) and incubated at room temperature for 5-minutes. Replaced with hybridization buffer (Stellaris) containing 5 μM 5′-Cy5-Oligo d(T)20 (Gene Link) and incubated in the dark for 16-hours to probe polyadenylated mRNA. Transferred to Wash Buffer A, placed at 37° C. for 30-minutes, then replaced with Wash Buffer B, incubating at room temperature for another 5-minutes. Washed 3× with PBS and imaged.

Western Blot to Assess G3BP1/2 Levels and Knockout Human Cells

U2OS WT, U2OS G3BP1/2 KO, HEK293, or HeLa cells from a 6-well plate were washed, trypsinized, quenched with media and centrifuged at 500×g for 5-minutes. Cell pellets were washed with PBS and flash-frozen. Immediately prior to lysis, cells were thawed on ice and resuspended in 150 μL 2× Nuage® LDS Sample Buffer/Reducing agent, sonicated, and boiled at 100° C. for 5-minutes. 50 ng of the following recombinant proteins were used with cell lysates as positive controls: G3BP1 (Novus, NBP1-50925-50UG), G3BP2 (Novus, NBP1-78843-100UG).

Samples were run on a NuPAGE® Novex 10% Bis-Tris Gel and transferred to PVDF Pre-Cut Blotting Membranes, as per manufacturer's protocol. Membranes were blocked overnight at 4° C. with rocking in 5% NFDM in TBST (5 mM Tris-HCl, pH 7.5, 15 mM NaCl, 1% Tween-20). Membranes were probed with the following primary antibodies in blocking solution overnight at 4° C. with rocking: G3BP1 (Mouse monoclonal, AbCam ab86135, 1:300), G3BP2 (Rabbit polyclonal, Abcam ab86135, 1:5000), Beta actin (Rabbit polyclonal, AbCam ab8227, 1:10,000). The next day, membranes were washed multiple times and then incubated with the following secondary antibodies in blocking solution for 30-minutes at room temperature with rocking: Peroxidase-AffiniPure Goat Anti-Mouse IgG (H+L) (Jackson, 115-035-062, 1:10,000), Peroxidase-AffiniPure Goat Anti-Rabbit IgG (H+L) (Jackson, 115-035-144, 1:10,000). Subsequently, multiple washes were performed prior to developing the membrane using SuperSignal™ West Pico PLUS Chemiluminescent Substrate, as per manufacturer's instructions.

Immunoprecipitation

150 mm dishes of near-confluent cells were treated as indicated, washed with cold Hanks Basic Salt Solution, and scrape-harvested at 4° C. into lysis buffer (20 mM Tris-HCl pH 7.4, 150 mM NaCl, 5 mM MgCl2, 1 mM DTT 0.5% NP-40, 10% glycerol) containing 1 mM DTT, protease inhibitors (Roche EDTA free), HALT phosphatase inhibitors (Pierce), and 20 μg/nL RNAse A. Cells were rotated for 30-minutes at 4° C., cleared by centrifugation (5,000 rpm for 5 minutes), and supernatants removed then incubated with Chromotek-GFP-Trap® Beads (Allele Biotech) for 2-hours with continuous rotation at 4° C. Beads were washed 5-times, and either eluted directly into SDS-lysis buffer with RNase treatment, or extracted in RIPA buffer (50 mM TRIS, 150 mM NaCl, 1.0% NP40, 0.5% DOC, 0.05% SDS) for 1-hour at 4° C. with rotation. Material released by RIPA buffer was recovered and precipitated with 60% acetone. Beads post-RIPA extraction contained bound material denoted “high affinity”, which was released by heating in reducing SDS-PAGE lysis buffer. Proteins were resolved on 4-20% Mini-PROTEAN TGX Precast Gel (Bio-Rad) and transferred to nitrocellulose membranes using the Transfer-Blot Turbo transfer system (Bio-Rad), and blotted using standard procedures as above. Chemiluminescence was detected using SuperSignal West Pico substrate (Thermo Scientific).

Cas9 Deletion Cell Lines

Each target sequence was purchased as paired DNA oligos (sense/antisense pairs) from IDT, annealed, and ligated into pCas-Guide (Origene), with the exception of UBAP2. Plasmid inserts were verified by sequencing, and cotransfected into cells with pDonor-D09 (Origene) encoding puromycin resistance. Following transfection, cells were subjected to a brief (24-hours) selection in puromycin (2 μg/mL) and allowed to recover for 2-days or longer before evaluation using the indicated antibodies and immunofluorescence. Cells were cloned by limiting dilution and clones were verified using both immunostaining and western blotting. For single KO lines, the parental cell line was U2OS expressing the tet-repressor (Kedersha et al 2016). CAPRIN1 and USP10 were individually knocked out in the previously characterized double (G3BP1/G3BP2) KO cells (Kedersha et al 2016).

To create the U2OSΔFFF cell line, FXR2 was first knocked out, clones were selected, and FXR2 protein expression was evaluated by immunofluorescence and Western blotting. Clone 6 was then co-transfected guide RNAs targeting FXR1 and FMR1. Clones were selected and screened in a similar manner and finally a triple-null line was obtained. All loci were sequenced to confirm deletions in the DNA.

In the case of UBAP2/URAP2L double KO, validated UBAP2L single-KO cells were plated into 200 μL of pCRISPRv2-UBAP2 gRNA (pooled, 6 gRNAs) or 200 μL of pCRISPRv2-Nontarget gRNA (Shalem et al., 2014) in 96-well plate. 72-hours later, confluent cells were washed, trypsinized and passaged into new wells containing 200 μL of the same lentivirus.

Cells were passaged three times and examined for successful KO, validating with two antibodies against UBAP2, which indicated that ˜30 percent of the cells feature very low or undetectable levels of UBAP2 (in NonTarget control, 100% of cells displayed UBAP2 staining). Cells were amplified by successive passage from 96-well to 24-well to 96-well over a 1-week period. Upon confluency in 96-well, cells were passaged at limiting dilution into 3 separate 96-well plates, so that each well featured ˜50% chance of receiving a cell. 10-days later, colonies were apparent in ˜20-30% of wells. For Nontarget control, six wells were harvested and passaged; candidate UBAP2/2L double-KOs, 50 separate lines.

Following ˜two weeks of additional passage and growth, candidate KO lines (and NonTarget controls) were plated onto fibronectin covered glass (96-well plate). 24-hours later, cells were at ˜60-80% confluency. Cells were fixed with 4% PFA, permeabilized with ice cold methanol for 5 minutes and immunohistochemistry was performed (anti-UBAP2, anti-G3BP1). In NonTarget controls, most cells featured G3BP-positive stress granules but they were slightly smaller than control conditions (i.e. non-UBAP2L KO), a result that was validated across labs (data not shown). 4 candidate UBAP2/2L double KO lines featured undetectable UBAP2 by immunofluorescence. In these examples, G3BP-positive SGs were only present in ˜30% of cells and they were much smaller in size than in WT or UBAP2L single-KOs. Double knockout of UBAP2 and UBAP2L was verified by Western blot.

Genotyping of Cas9 Mutant Cell Lines

To identify Cas9-induced mutations of all KO cell lines in the coding sequence, genomic amplification was performed using nested primer sets surrounding the region targeted by the particular guide sequence. Genomic DNA PCR was done with Invitrogen's AccuPrime GC-Rich DNA Polymerase (Buffer A). DNA was initially denatured at 95° C. for 3-minutes, followed by denaturation at 95° C. for 30-seconds, annealing at 60° C. for 30-seconds, and extension at 72° C. for 1-minute for 30 cycles. Final extension was done at 72° C. for 10-minutes. PCR amplicons were directly sequenced. If there was evidence for multiple sequences (i.e. multiple alleles), PCR products were adenylated using Taq polymerase and cloned into Promega pGEM®-T Easy vector; individual clones were obtained and sequenced.

Double-Positive U2OS Stable Cell Lines

A clonal cell line was made constitutively expressing mCherry-G3BP1 by transfection of mCherry-G3BP1-C1 into the (G3BP1/G3BP2) KO cells containing the tet repressor, selected using G418 (500 μg/mL), and cloned. This line was used to make double-positive cells expressing tet-inducible GFP-tagged proteins (G3BP1 WT, G3BP1 S38F, G3BP1 F33W, and UBAP2L WT) in pcDNA4 t/o vector (Invitrogen), selected using zeocin (Invtrogen, 250 μg/mL).

Quantification and Statistical Analysis

Fluorescence Correlation Spectroscopy

GFP and mCherry fluorescence values were converted to absolute concentrations using fluorescence correlation spectroscopy (FCS), performed as described previously (Bracha et al., 2018) with minor modifications. Data for diffusion and concentration of indicated fluorescent fusion proteins were obtained with 30-second FCS measurement time.

The measurements were performed on U2OS G3BP1/2 double-KO cell populations separately expressing iLID-mGFP and mCherry-sspB, fusion protein conditions that were chosen based on the assumption that such non-native fusion proteins would be monomeric and feature no major endogenous binding partners. Images were taken using a Nikon A1 laser scanning confocal microscope with an oil immersion objective (Plan Apo 60×/1.4 numerical aperture, Nikon). All measurements and data analysis were performed using the SymPhoTime Software (PicoQuant).

The autocorrelation function for simple diffusion is:

$$G ( τ ) = G ( 0 ) {( 1 + ( \frac{τ}{{τ}_{D}} ) )}^{- 1} {( 1 + ( \frac{τ}{{κ}^{2} {τ}_{D}} ) )}^{- 0.5}$$

The variables in the above equation are defined as follows: G(0) is magnitude at short time scales; τ is the lag time; τ_{D} is the half decay time; and κ is the ratio of axial to radial of measurement volume

$$κ = ( \frac{{ω}_{z}}{{ω}_{x y}} ) .$$

Here, ω_{xy}=0.19 μm and κ=5.1, which is determined by the fluorophore dye Alexa 488 in water. The parameters τ_{D} and G(0) are optimized in the fit and are used to determine the diffusion coefficient (D=ω_{xy}²/4τ_{D}) and molecule concentration

$$( C = {( {π}^{\frac{3}{2}} {ω}_{xy}^{2} {ω}_{z} G ( 0 ) )}^{- 1} ) .$$

The fluorescence to concentration calibration curves displayed in FIG. 18 were used for all experiments that quantitatively approximated the concentrations of mCherry- and mGFP-tagged fusion proteins in WT and G3BP KO U2OS cells. Such FCS calibration curves yielded several findings that supported the precision of such estimates:

(A) Independently performed mCherry FCS experiments yielded concentration estimates that were <5% different (Bracha et al., 2018). Further, the aforementioned study used an auto-catalytic P2A system to co-express mGFP and mCherry at equimolar ratio, with GFP concentrations extrapolated from the FCS calibration curves determined for mCherry. This indirectly extrapolated calibration curve predicted GFP concentrations that differed by <20% from the independently obtained calibrations and estimations used in this study.

(B) Slope that quantifies stoichiometry between USP10 and G3BP required for rescue of G3BP defects, was remarkably close to 1 (˜0.98), the predicted value for such a competitive inhibitor expressed at concentrations far greater than its Kd and is further confirmed by nearly equivalent slope for different strong inhibitors (USP10 NIMx1 and CAPRIN1 1-386);

(C) the concentration of G3BP1/2 in U2OS cells were extrapolated by adding the G3BP concentration for rescue (620 nM) (values ascertained in separate experiments were within 50 nM of this value) and USP10 for SG inhibition (1560 nM) to extrapolate a concentration of ˜2180 nM G3BP in the cytoplasm of U2OS cells. This value is approximately equal to independently obtained mass spectrometry values in HeLa cells (Hein et al., 2015) and Western blot confirms similar levels between the two cell lines;

(D) mGFP-G3BP1 and G3BP1-mCherry feature very similar SG rescue concentration thresholds (i.e., within 50 nM of each other).

Image Analysis

All images were analyzed using a combination of manual image segmentation (ImageJ), custom semi-automated workflows in ImageJ, and MATLAB 2018b. In all experiments, regions of interest were selected in ImageJ and average cytoplasmic intensities were calculated using the aforementioned FCS calibration. The presence of stress granules was, in cases other than the cycling experiments, determined by manual scoring based upon co-localization with a marker of stress granules that features diffuse distribution in the cytoplasm in the absence of stress.

Manual Image Segmentation

The average fluorescence intensity for mCherry and mGFP for a cell was used to approximate the concentration of associated fusion proteins. This was determined by using manual image segmentation to draw 4.5×4.5 μm square ROIs in cytoplasmic regions featuring homogenous distribution of fluorescence (i.e. regions with low density of membrane-bound organelles like the endoplasmic reticulum). The aforementioned FCS calibration curves were then used to determine the protein's concentration. Presence or absence of stress granules was manually annotated for experiments not involving Corelets. For the purpose of phase diagrams, phase separation was manually annotated by assessing whether macroscopic puncta formed following a 5-minute activation time course (6-second intervals). Only fully activated cells were considered to avoid confounds related to diffusion-based capture (Bracha et al., 2018).

Light-Dark Cycling Experiments

Individual regions of interest, which remained in the field of view throughout the time lapse were manually selected. Standard deviations were calculated from the measured mCherry intensity and were normalized by the standard deviation at the first frame taken.

G3BP Rescue Competition Data Analysis in G3BP KO U2OS Cells

The concentration of each cell was determined via manual image segmentation as previously described and absence or presence of stress granules was annotated. To determine a boundary from the data, a support vector machine (SVM) trained using the concentrations of the two components as explanatory variables and the categorical stress granule state as a response variable by applying the fitcsvm( ) function in the MATLAB Statistics and Machine Learning package using the default solver. Briefly, a support vector machine constructs a linear decision surface based on boundary points (‘support vectors’), with the assumption that the data is linearly separable. In this two-dimensional case, the parameters of slope and intercept were extracted to calculate the minimal G3BP concentration for stress granule formation as well as the stoichiometry of interactions with proteins of interest.

Phase Diagrams and Calculation of Critical Valence

For each phase diagram, mean concentrations of both iLID-GFP-Fe core and mCherry-sspB-tagged proteins were calculated and assigned to the category of having or not having stress granules. To determine phase threshold boundaries in an automated and unbiased fashion, an SVM regressor was again used, using the core concentration and log 2-transformed valence as explanatory variables with the presence of phase separated structures as a categorical response variable. However, because the data was not linearly separable, a polynomial kernel with degree 2 was used to account for the curvature of the phase threshold. Then, to calculate the decision surface, the score of the SVM was calculated at all points in a 50 by 50 grid in the phase diagram, and a contour line representing the phase threshold was drawn connecting points with a score of 0 using MATLAB's contour( ) function. Specific values for critical valence at specified core concentrations were then calculated by linearly interpreting the zero-score contour line.

Approximation of the critical concentration for inhibition of stress granule assembly in WT U2OS cells

For each experiment, the concentration of the protein of interest was determined for each cell, and the presence of stress granules was categorized. The critical concentration of inhibition or rescue was defined as the concentration of protein of interest at which cells had a 50 percent chance of having stress granules. Specifically, the probability density was calculated by binning the concentration distribution using a square root number rule. Within each bin, the probability of having stress granules was calculated as the number of cells with stress granules over the total number of cells in that bin. This results in a monotonic function; its value at a probability of 0.5 was then interpolated to determine the threshold concentration of inhibition or rescue. This was repeated for each replicate and standard error of the mean between replicates used to determine error bars.

1. A protein system comprising:
one or more optoproteins, each optoprotein comprising a first region fused to a second region, the first region wising at least one light sensitive protein or cognate partner of a light sensitive protein and one or more proteins configured to self-assemble, and the second region comprising one or more folded RNA binding domains (RBDs), disordered RBsDs, folded non-RBD domains, or combination thereof.

2. The protein system according to claim 1,
wherein the first region of the optoprotein comprises a first cognate partner of a first light sensitive protein, and
wherein the protein system further comprises:
a core protein comprising a first region fused to a second region, the first region of the core protein comprising the first light sensitive protein, and the second region of the core protein comprising one or more folded RNA binding domains (RBDs), disordered RBDs, folded non-RBD domains, or combination thereof the second region of the core protein being adapted to self-assemble.

3. The protein system according to claim 2, wherein the protein system further comprises:
a fixed linker protein comprising a first region fused to a second region, each first and second region of the fixed linker protein comprising one or more folded RNA binding domains (RBDs), disordered RBDs, folded non-RBD domains, or combination thereof, and each first and second region of the fixed linker protein being adapted to interact with the second region of the optoprotein.

4. The protein system according to claim 2,wherein the protein system further comprises:
an alternate optoprotein comprising a first region fused to a second region, the first region of the alternate optoprotein comprising a second cognate partner of a second light sensitive protein, the second region of the alternate optoprotein comprising one or more folded RNA binding domains (RBDs), disordered RBDs, folded non-RBD domains, or combination thereof, the second region of the alternate optoprotein adapted to interact with the second region of the first fusion protein; and
an alternate core protein comprising a first region fused to a second region, the first region of the alternate core protein comprising the second light sensitive protein, and the second region of the alternate core protein comprising one or more folded RNA binding domains (RBDs), disordered RBDs, folded non-RBD domains, or combination thereof, the second region of the alternate core being adapted to self-assemble.

5. The protein system according to claim 1,
wherein the system comprises at least two optoproteins, wherein one of the at least two optoproteins comprises a light sensitive protein, and wherein another of the at least two first optoproteins comprises a cognate partner of the light sensitive protein
wherein the system further comprises two or more PPI core proteins, each PPI core protein comprising a first region fused to a second region, the first region of the PPI core protein comprising one or more folded RNA binding domains (RBDs), disordered RBDs, folded non-RBD domains, or combination thereof, the second region of the PPI core protein comprising one or more folded RNA binding domains (RBDs), disordered RBDs, folded non-RBD domains, or combination thereof, and
wherein the first region of each PPI core protein adapted to interact with the second region of the optoproteins, and wherein the second region of each PPI core protein is adapted to self-assemble.

6. The protein system according to claim 1,
wherein the first region of the optoprotein comprises a light sensitive protein, and
wherein the protein system further comprises:
an additional optoprotein, wherein the first region comprises the light sensitive protein, and the second region comprises one or more folded RNA binding domains (RBDs), disordered RBDs, folded non-RBD domains, or combination thereof, wherein the second region of the additional optoprotein is adapted to interact with the second region of the optoprotein and
wherein the first region of the optoprotein and the first region of the additional optoprotein are adapted to self-assemble in response to light into an oligomer of at least 2.

7. The protein system according to claim 1, wherein the light-sensitive protein is fused to a folded RBD, and the folded RBD is an RNA recognition motif (RIM), a K homology (KH) domain, a Pumilio (PUM) domain, a zinc-finger domain, a DEAD box helicase domain, a double-stranded RNA-binding domain (dsRBD), an m6A RNA-binding domain (YTH domain), or a Cold shock domain (CSD).

8. The protein system according to claim 1, wherein the light-sensitive protein is fused to a disordered RBD, and the disordered RBD is an arginine-glycine (RG) domain, an arginine-glycine-glycine (RGG) domain, a serine-arginine (SR) domain, or a basic-acidic dipeptide (BAD) domain.

9. The protein system according to claim 1, wherein the light-sensitive protein is fused to one or more folded non-RBDs.

10. The protein system according to claim 1, wherein the one or more proteins configured to self-assemble comprises ferritin.

11. The protein system according to claim 1, wherein the at least one light-sensitive protein is an engineered protein.

12. The protein system according to claim 11, wherein the engineered protein is LOV2-ssrA.

13. The protein system according to claim 1, wherein the first region comprises two LOV2-ssrA proteins.

14. The protein system according to claim 1, wherein at least one fusion protein comprises a fluorescent tag.

15. A cell line or a stern cell-derived cell that expresses the protein system of claim 1.

16. The cell line or a stern cell-derived cell according to claim 15,
wherein one or more genes configured to express the protein system are delivered to the cells utilizing a lentivirus, an adeno-associated virus (AAV), bacterial artificial chromosomes (BAC), transient transfection (e.g. liposomes or proprietary formulations for DNA plasmid introduction), micro-injection, electroporation, or a CRISPR/Cas9-based approach.

17. The cell line or a stem cell-derived cell according to claim 15, comprising human cells, yeast cells, cultured neurons, or cells from worm, fly, rodent, or primate models.

18. An expression vector system, the expression vector system comprising at least one expression vector configured to transfect a cell with one or more genes configured to express the protein system according to claim 1.

19. The expression vector system according to claim 18, wherein the expression vector system comprises a first plasmid comprising a gene capable of expressing the first fusion protein.

20. A method for measuring phase behavior of natural or engineered multi-component condensates, comprising the steps of:
a. providing a protein system according to claim 1;
b. oligomerizing the folded RNA binding domain RBD, disordered RBD, or folded non-RBD domains by exposing the light-sensitive protein to at least one wavelength of light; and
c. measuring phase behavior by mapping a phase diagram, determining if phase separation, condensation, or aggregation occurs, measuring a condensate material property, a protein concentration, a valence, or a combination thereof.

21. The method according to claim 20, wherein the protein system is located within a living cell.

22. The method according to claim 20, wherein the protein system is located outside a living or dead cell.

23. The method according to claim 20, wherein oligomerization drives gelation of a cystoplasmic ribonucleoprotein (RNP) granule.

24. The method according to claim 20, wherein the protein system is in a well in a multi-well array/plate.

25. The method according to claim 24, further comprising providing one or more chemical agents to the well.

26. The method according to claim 20, further comprising utilizing a genetic screen based on gene knockdown or gene upregulation.

27. The method according to claim 20, further comprising:
determining the impact a genetic screen based on gene knockdown, a genetic screen based on upregulation, the addition of one or more chemical agents to a well, or a combination thereof has, based on the measured phase behavior.

28. A method of inducing stress granule formation comprising:
a. providing a protein system comprising one or more optoproteins, each optoprotein comprising a first region fused to a second region, the first region comprising at least one light sensitive protein or cognate partner of a light sensitive protein and one or more proteins configured to self-assemble, and the second region comprising one or more folded RNA binding domains (RBDs), disordered RBDs, folded non-RBD domains, or combination thereof; and
b. oligomerizing the folded RNA binding (RBD), disordered RBD, or folded non-RBD domains by exposing the light-sensitive protein to at least one wavelength of light; wherein the oligomerization induces stress granule formation.

29. A protein system comprising:
one or more optoproteins, each optoprotein comprising a first region fused to a second region, the first region comprising a first cognate partner of a first light sensitive protein, the second region comprising one or more folded RNA binding domains (RBDs), disordered RBDs, folded non-RBD domains, or a combination thereof; and
a core protein comprising a first region fused to a second region, the first region of the core protein comprising the first light sensitive protein, and the second region of the core protein comprising one or more folded RNA binding domains, disordered RBDs, folded non-RBD domains, or combination thereof, the second region of the core protein being adapted to self-assemble.

30. The protein system according to claim 29,
wherein the protein system further comprises:
a fixed linker protein comprising a first region fused to a second region, each first and second region of the fixed linker protein comprising one or more folded RNA binding domains (RBDs), disordered RBDs, folded non-RBD domains, or combination thereof, and each first and second region of the fixed linker protein being adapted to interact with the second region of the optoprotein.

31. The protein system according to claim 29,
wherein the protein system further comprises:
an alternate optoprotein comprising a first region fused to a second region, the first region of the alternate optoprotein comprising a second cognate partner of a second light sensitive protein, the second region of the alternate optoprotein comprising one or more folded RNA binding domains (RBDs), disordered RBDs, folded non-RBD domains, or combination thereof, the second region of the alternate optoprotein adapted to interact with the second region of the first fusion protein; and
an alternate core protein comprising a first region fused to a second region, the first region of the alternate core protein comprising the second light sensitive protein, and the second region of the alternate core protein comprising one or more folded RNA binding domains (RBDs), disordered RBDs, folded non-RBD domains, or combination thereof, the second region of the alternate core being adapted to self-assemble.

32. A protein system comprising:
at least two optoproteins, each optoprotein comprising a first region fused to a second region, the first region comprising at least one light sensitive protein or cognate partner of a light sensitive protein, and the second region comprising one or more folded RNA binding domains (RBDs), disordered RBDs, folded non-RBD domains, or combination thereof;
wherein one of the at least two optoproteins comprises the light sensitive protein, and wherein another of the at least two first optoproteins comprises the cognate partner of the light sensitive protein; and
two or more protein-protein interaction (PPI) core proteins, each core protein comprising a first region fused to a second region, the first region of the PPI core protein comprising one or more folded RNA binding domains (ABDO, disordered RBDs, folded non-RBD domains, or combination thereof, the second region of the PPI core protein comprising one or more folded RNA binding domains (RBDs), disordered RBDs, folded non-RBD domains, or combination thereof;
wherein the first region of each PPI core protein is adapted to interact with the second region of the optoproteins, and wherein the second region of each PPI core protein is adapted to self-assemble.

33. A protein system comprising:
a first optoprotein comprising a first region fused to a second region, the first region s a first light sensitive protein, the second region comprising one or more folded RNA binding domains (RBDs), disordered RBDs, folded non-RBD domains, or a combination thereof; and
an additional optoprotein comprising a first region fused to a second region, the first region comprising the first light sensitive protein, and the second region comprising one or more folded RNA binding domains (RBDs), disordered RBDs, folded non-RBD domains, or combination thereof,
wherein the second region of the additional optoprotein is adapted to interact with the second region of the first optoprotein; and
wherein the first region of the first optoprotein and the first region of the additional optoprotein are adapted to self-assemble in response to light into an oligomer of at least 2.
